SEQUENCE LISTING (1 sequence)

SEQ ID NO: 1 - protein, 14 aa, Artificial Sequence
  PKC specific substrate
RRGRTGRGRRGIFR

(19) United States
(12) Patent Application Publication
(10) Pub. No.: US 2010/0190187 A1
(43) Pub. Date: Jul. 29, 2010

(54) Screening Assays for Antagonists and Analyses of Cardiac Hypertrophy

(75) Inventors: Robert U. Simpson, Ann Arbor, MI (US); Daniel Tishkoff, Ypsilanti, MI (US); Karl Nibbelink, Ann Arbor, MI (US)

Correspondence Address: HARNESS, DICKEY & PIERCE, P.L.C., P.O. BOX 828, BLOOMFIELD HILLS, MI, 48303, US

(73) Assignee: The Regents of the University of Michigan Office of Technology Transfer, Ann Arbor, MI (US)

(21) Appl. No.: 12/224,004

(22) PCT Filed: Feb. 14, 2007
(86) PCT No.: PCT/US07/03878

§ 371 Date: Aug. 14, 2008

Related U.S. Application Data
(60) Provisional application No. 60/773,511, filed on Feb. 15, 2006.
(60) Provisional application No. 60/877,055, filed on Dec. 22, 2006.

(51) Int. Cl. G01N 33/53; C12Q 1/48; C12Q 1/02

(52) U.S. Cl. 435/7.6; 436/501; 435/15; 435/29

(57) ABSTRACT

Various embodiments of the present invention provide methods for screening for candidate heart failure compounds employing screening assays effective in identifying agonists or antagonists or ligands of vitamin D receptor mediated pathways implicated in heart failure. Methods are provided for the screening of test compounds that can specifically bind to the vitamin D receptor. Methods for screening for a test compound which modulates the activity of a VDR for the treatment of heart failure, wherein the method comprises: (a) contacting a test compound with VDR in a reaction mixture, wherein the reaction mixture conditions permits the test compound to bind to a VDR, including membrane VDR and nuclear VDR. The binding between the test compound and the VDR is compared to a reference such as Vitamin D3. The modulation of biomarkers after a test compound has bound and activated a VDR are also measured and compared to samples in the absence of test compound.

### CROSS-REFERENCE TO RELATED APPLICATIONS

This application claims priority to U.S. Ser. No. 60/773,511 filed Feb. 15, 2006 and U.S. Ser. No. 60/877,055 filed Dec. 22, 2006. Each of the above references is incorporated herein by reference in its entirety.

### GOVERNMENT RIGHTS

This disclosure was made with government support under National Institutes of Health Grant No. R01-HL074894. The Government has certain rights in the invention.

### FIELD

The present disclosure relates to the use of cultured and isolated cardiomyocytes and cells having a vitamin D receptor for cell based screening assays to study cardiac hypertrophy, including assays developed to screen agonist and antagonist compounds effective in modulating cellular events leading to cardiac hypertrophy (heart failure) including vitamin D receptor signaling events.

### BACKGROUND

The statements in this section merely provide background information related to the present disclosure and may not constitute prior art.

Heart failure is a major and growing public health problem in the United States. Approximately 5 million patients in the U.S. have heart failure, and over 550,000 patients are diagnosed with heart failure for the first time each year. The disorder is the primary reason for 12 to 15 million office visits and 6.5 million hospital days each year. In 2001, nearly 53,000 patients died of heart failure as a primary cause. The number of deaths related to heart failure has steadily increased despite advances in better treatments.

Heart failure is a complex disease of multiple etiologies that lead to progressive cardiac dysfunction. The clinical syndrome of heart failure may result from disorders of the pericardium, myocardium, endocardium, or great vessels, but the majority of patients suffering from heart failure have symptoms associated with impairment of left ventricle myocardial function. Left ventricle dysfunction begins with some injury to or stress on the myocardium and is generally progressive, despite any identifiable insult to the heart. The key feature of the initial dysfunction of the myocardium is evidenced by changes in the geometry and structure of the left ventricle, such that the chamber dilates and/or hypertrophies and becomes more spherical, a process called cardiac remodeling. Cardiac remodeling is thought to precede symptoms, continues after appearance of symptoms and contributes to the worsening of symptoms despite treatment.

Despite studies and treatments for heart failure targeting the neurohormonal system and its cognate factors implicated in heart failure, there is a great need to understand the biology of the dysfunction and dysregulation of the cardiac myocyte itself. Many neurohormonal system factors have specific roles outside of the contractile cardiac myocyte itself, such as regulation of blood pressure and sodium retention, conditions that may affect the remodeling of the cardiomyocyte itself, albeit somewhat indirectly. A greater understanding of cell surface membrane receptors and their cognate intracellular mechanisms occurring within the cardiac myocyte is needed. Once these mechanisms are elucidated, further treatment options can be explored.

Prior studies have shown that, Vitamin D exerts one of its many effects by impairing the ability of cultured neonatal ventricular myocytes to mature. T.O'Connell et al., Endocrinology 136(2):482-488 (1995). Further evidence linking the direct effects of Vitamin D to cardiac function include data provided by T.O'Connell et al., Biochem. Biophys. Res. Commun. 213(1): 59-65 (1995) showing that Vitamin D administration to rats inhibits proliferation of cardiomyocytes along with reduced levels of the oncogene c-myc. Many of the roles of Vitamin D on heart function were initially shown in animals provided with Vitamin D deficient diets. These animals were shown to have increased myocardial contractility and were found to have cardiac hypertrophy. Weishaar, W. et al., J. Clin. Invest. 79(6):1706-1712, (1987).

The development of screening assays for test compounds that can affect the cellular mechanisms that cause left ventricle remodeling and calcium dysregulation in the cardiomyocyte is therefore of great importance. Furthermore, screening assays for agonists of membrane and nuclear VDR receptors in the cardiomyocyte presents an interesting target for potential therapeutic agents. However, evidentiary support of the role of secondary messengers and membrane receptor activity in the expression and development of heart failure have mostly come from experiments conducted with live and transgenic animals. As a result, it would be desirable to provide effective screening assays that are able to identify compounds capable of binding and activating Vitamin D receptors and improve contractility by correcting calcium utilization in the cardiomyocyte.

### SUMMARY

Various embodiments of the present invention provide methods for screening for candidate heart failure compounds employing screening assays effective in identifying agonists or antagonists or ligands of vitamin D receptor mediated pathways implicated in heart failure. Methods are provided for the screening of test compounds that can specifically bind to the vitamin D receptor. A method for screening for a test compound which modulates the activity of a VDR for the treatment of heart failure, wherein the method comprises: (a) contacting a test compound with VDR in a reaction mixture, wherein the reaction mixture conditions permits the test compound to bind to a VDR, including membrane VDR and nuclear VDR. The binding between the test compound and the VDR is compared to a reference such as Vitamin D3. The modulation of biomarkers after a test compound has bound and activated a VDR are also measured and compared to samples in the absence of test compound. Test compounds that can bind specifically to VDR (both membrane VDR and nuclear VDR) and modulate one or more biomarkers associated with cardiac hypertrophy are then identified as candidate heart failure compounds.

### DRAWINGS

The drawings described herein are for illustration purposes only and are not intended to limit the scope of the present disclosure in any way.

FIG. 1 illustrates various Vitamin D analogs useful in various embodiments accordance with the present disclosure;

FIG. 2 schematically represents the metabolic pathway involving Vitamin D and synthesis of active metabolites 1,25-Dihydroxyvitamin D₃ and 24,25-Dihydroxyvitamin D₃.

FIGS. 3(A)-(D) depicts confocal micrographs of immunofluorescence staining of rat cardiomyocytes with anti-VDR antibodies. In FIG. 3(A) depicts a high magnification of rat cardiomyocyte stained with anti-VDR antibodies, FIG. 3(B) Lower magnification of FIG. 3A, cells stained with primary antibody sc 13133 (anti-VDR D6) also showing diffuse VDR staining on the membrane and also in the t-tubule membrane structures. FIG. 3C) peptide blocked cell illustrating that the staining by the primary antibody sc 13133 (anti-VDR D6) is specific to VDR located in the membrane and t-tubules. FIG. 3D) cells were incubated only with the secondary antibody showing the absence of non-specific binding of the secondary antibody.

FIGS. 4(A)-4(C) depict photomicrograph immunofluorescence showing staining of rat cardiomyocytes with anti-SERCA2 antibodies. In FIG. 4(A), nuclei was stained with DAPI (FIG. 4A) cells treated with primary antibody sc2783 (anti-SERCA2 C6) showing the localization of SERCA2 to the membrane and transverse tubules. Secondary antibody conjugated to Texas Red (FIG. 8B) cells treated with primary antibody sc2783 (anti-SERCA2 C6) showing the localization of SERCA2 to the membrane and transverse tubules. FIG. 4C depicts secondary antibody conjugated to FITC alone in the absence of primary antibody illustrating that the staining by the primary antibody sc2783 (anti-SERCA2 C6) is specific to SERCA2.

FIGS. 5(A)-5(C) depict photomicrograph staining of rat cardiomyocytes with anti-VDR and anti-SERCA2 and DAPI. In FIG. 5A-5C nuclei are stained with DAPI. FIG. 5A depicts cells stained with anti-VDR antibody using a laser at a wavelength to visualize FITC staining throughout the cell. VDR is localized to the membrane and the t-tubules. FIG. 5B depicts cells stained with the primary antibody anti-SERCA2 using a laser set at a wavelength to visualize Texas Red staining throughout the cell. SERCA2 is localized to the membrane and the t-tubules. FIG. 5C depicts the addition of antibodies to both VDR and SERCA2. These antibodies are reacted with secondary antibodies labeled with different fluorescence markers and can be concurrently visualized using two lasers at different wavelengths to indicate that the VDR and SERCA2 are co-localized to the same cellular locations, i.e., the membrane and the t-tubules of rat cardiomyocytes.

FIG. 6 Competition experiment showing the relative ability of non-radiolabelled 1,25 dihydroxy vitamin D₃ and Vitamin D₅ to compete with radiolabeled [³H] 1a,25(OH)₂ vitamin D₃ for binding to mVDR in membrane preparations derived from rat whole heart homogenates.

FIG. 7 Comparison of the relative ability of select 1,25 dihydroxy vitamin D₃ analogs to compete with radiolabeled [³H] 1a,25(OH)₂ vitamin D₃ binding to the mVDR in membrane preparations derived from rat whole heart homogenates.

FIG. 8 Specific binding of [³H] 1α,25(OH)₂ vitamin D₃ to plasma membrane fractions (7,700-40,000×g) isolated from rat whole heart homogenates. Scatchard transformation of the primary data shows that the specific binding of [³H] 1α,25(OH)₂ vitamin D₃ to mVDR has a Bₘₐₓ of 186 fmol/mg and a K_{d} of 695.8 pM. nuclei were stained with DAPI (FIG. 7A) cells treated with primary antibody sc13133 (anti-VDR D-6) showing the localization of membrane bound VDR to the membrane and transverse tubules 100×.

FIG. 9 Western Blot analysis of VDR localization in fractionated whole rat heart homogenates. Rat whole heart homogenates were fractionated by differential centrifugation (0-7,700 g nuclear/mitochondrial fraction, 7,700-40,000 g plasma membrane fraction, 40,000-110,000 g microsome fraction and supernatant cytosol fraction) and samples (100 μg) were separated by SDS-PAGE and transferred onto PVDF. The blot was then probed for the presence of VDR using anti-VDR (sc1008 C-20). The bulk of the VDR was localized to the plasma membrane (7,700-40,000×g) and cytosol fractions.

FIGS. 10(A) & 10(B) depicts the physiological effect of Vitamin D3 on cellular growth and proliferation. FIG. 10(A) illustrates the dose dependent inhibition of cellular proliferation when cells are incubated with a test compound (Vitamin D); FIG. 10(B) illustrates the optimal inhibitory concentration of a test compound (Vitamin D);

FIG. 11 illustrates an expression of a cardiac-specific phenotype in the HL-1 cardiomyocyte cell using light microscopic images of HL-1 cells incubated with and without a test compound (Vitamin D3) compared to controls;

FIG. 12 (A)-(E) illustrates immunofluorescence staining of HL-1 cells treated and untreated with Vitamin D3 using antibodies to known hypertrophy related proteins;

FIG. 13(A)-(C) illustrates a western blot analysis of cells incubated in the presence and absence of a test compound (Vitamin D3); 13(A) depicts expression of VDR, ANP, myothrophin and c-myc relative to control when treated with 1α,25(OH)₂D₃. 13(B) illustrates an expression of VDR after 1 and 24 hours post exposure to Vitamin D3; and 13(C) illustrates a Vitamin D 3 dose dependent induction of expression of VDR in HL-1 cells.

### DETAILED DESCRIPTION

The following description is merely exemplary in nature and is not intended to limit the present disclosure, application, or uses

The present disclosure relates to methods for screening for test compounds that can bind to the Vitamin D receptor (VDR) and inhibit or prevent cardiac hypertrophy. Such test compounds are candidate compounds for heart failure. Methods for screening include methods that identify compounds that modulate the activity of a membrane VDR (mVDR) wherein the candidate compound is capable of directly binding to the mVDR in the presence of a competitor or known Vitamin D molecule such as 1α,25 dihydroxy vitamin D3 (1α,25(OH)₂D₃. In another aspect, the present disclosure also provides for screening methods for test compounds that are capable of binding to mVDR and activate PKC phosphorylation of a specific Protein Kinase C substrate that is important for modulating calcium homeostasis or other factors implicated in normal cell structure, cardiomyocyte contraction, growth and differentiation. In still another aspect, methods for screening for target compounds for identifying candidate compounds for heart failure that are capable of binding to a VDR and increasing PKC activity. In still a further aspect, the present disclosure provides for screening assays that screen for test compounds that can alter the cardiomyocyte cell phenotype though activation of the VDR, i.e. the size, proliferation and morphology when a test compound is contacted with a cardiomyocyte. The present disclosure also provides for a method for screening for test compounds that are candidate heart failure compounds when the test compound can modulate the expression of a cardiac hypertrophy biomarker such as phospholamban phosphorylation, expression of c-myc, myotrophin, PCNA, calbindin 9 & 32, atrial natriuretic peptide (ANP) and other gene products that are specifically up-regulated or down-regulated as a result of the pathological processes that are involved in driving a normal cardiomyocyte to a hypertrophic cardiomyocyte.

### Modulation of Vitamin D Activity in the Cardiomyocyte

Several laboratories have characterized the effects that modification of the vitamin D endocrine system has on cardiac muscle structure and function. Previous studies have shown that vitamin D₃ deficiency alters rat myocardial morphology, extra cellular matrix (ECM) and function. Subsequent studies have revealed that large and statistically significant increases in ventricular pressure development (+dP/dt) are observed in perfused hearts from young (9-week old) vitamin D₃-deficient rats compared to age matched hearts from vitamin D₃-sufficient rats. A mechanism by which myocardial contractility can be increased is by raising intracellular calcium concentrations. 9-week-old vitamin D₃-deficient rats show an increase of L-type calcium channels and post rest contraction response, a measure of sarcoplasmic reticulum (SR) calcium uptake.

Reduced levels of the most active vitamin D metabolite, 1,25-dihydroxyvitamin D₃, are associated with an increased risk of heart failure and reports also indicate ventricular function is compromised and a dilated cardiomyopathy develops in pediatric patients with rickets caused by a vitamin D deficiency. Weishaar, W. et al., J. Clin. Invest. 79(6): 1706-1712, (1987).

### Mode of Action of Vitamin D

### Vitamin D

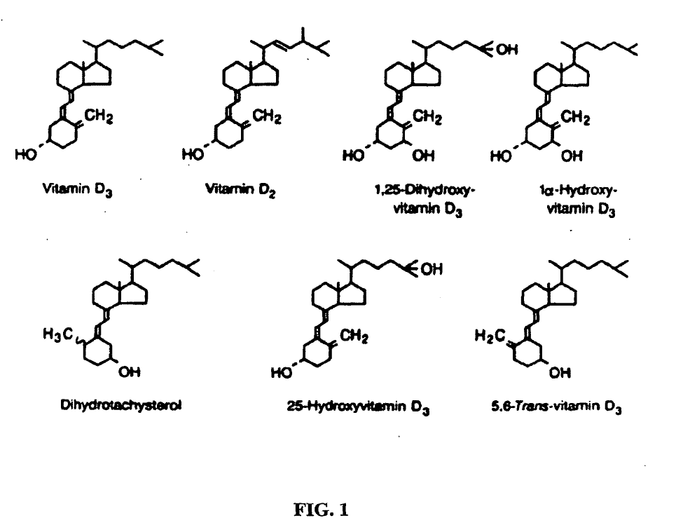

Vitamin D is essential for life in higher animals. It is one of the most important biological regulators of calcium metabolism. Along with the two peptide hormones, parathyroid hormone and calcitonin, vitamin D is responsible for the minute-by-minute, as well as the day-to-day, maintenance of calcium/mineral homeostasis. These important biological effects are achieved as a consequence of the metabolism of vitamin D into a family of metabolites. One of these metabolites, namely 1α,25(OH)₂-vitamin D_{3 [}1α,25(OH)₂D₃], is considered to be a steroid hormone as shown in FIG. 1

### Vitamin D Endocrine System

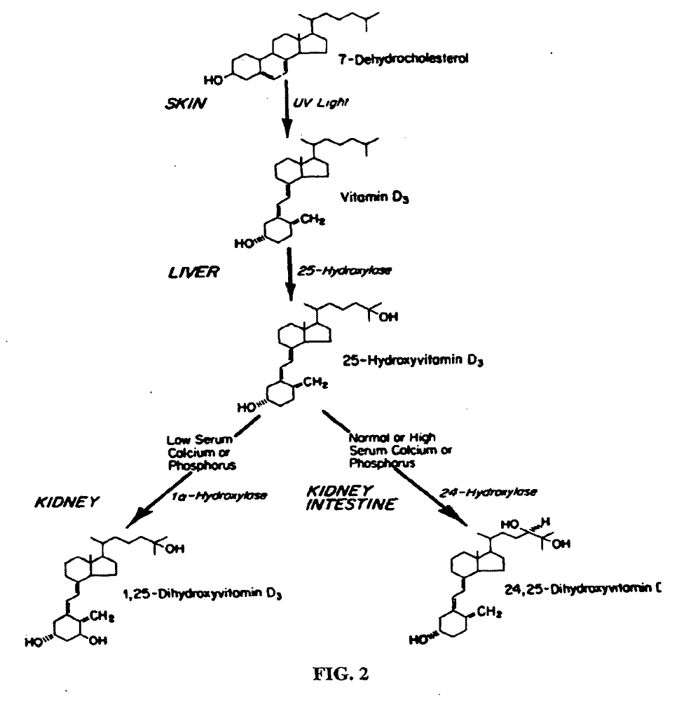

The scope of the biological responses related to vitamin D is best understood through the concept of the vitamin D endocrine system model as seen in FIG. 2. This model is based on the fact that vitamin D₃ is, in reality, a prohormone and is not known to have any intrinsic biological activity itself. It is only after vitamin D₃ is metabolized first into 25(OH)D₃ in the liver and then into 1α,25(OH)₂D₃ and 24R,25(OH)₂D₃ by the kidney, that biologically active molecules are produced. Today some 37 vitamin D₃ metabolites have been isolated and chemically characterized. This invention concerns biologically active analogs of 1α,25(OH)₂D₃.

The core elements of the vitamin D endocrine system include the skin, liver, kidney, blood circulation and other target organs. As shown in FIG. 2, photoconversion of vitamin D (7-dehydrocholesterol) to vitamin D₃ (activated 7-dehydrocholesterol) occurs in the skin. Alternatively, vitamin D₃ is supplied by the dietary intake. Vitamin D₃ is then metabolized by the liver to 25(OH)D₃, the major form of vitamin D circulating in the blood. The kidney, functioning as an endocrine gland, converts 25(OH)D₃ to the two principal dihydroxylated metabolites, namely 1α,25(OH)₂D₃ and 24R,25(OH)₂D₃. The hydrophobic vitamin D and its metabolites, particularly 1α,25(OH)₂D₃, are bound to the vitamin D binding protein (DBP) present in the plasma and systemically transported to distal target organs. 1α,25(OH)₂D₃ binding to the target organs cell receptors is followed by the generation of appropriate biological responses through a variety of signal transduction pathways.

### Vitamin D Receptors

The spectrum of biological responses mediated by the hormone 1α,25(OH)₂D₃ occurs as a consequence of the interaction of 1α,25(OH)₂D₃ with two classes of specific receptors. These receptors are identified as the nuclear receptor, (nVDR), and the cellular membrane receptor, (mVDR). The 1α,25(OH)₂D₃ nVDR from several species has been characterized both biochemically and genetically. The nVDR protein was determined to have a molecular weight of about 50 kDa. Through cloning, the nVDR was shown to belong to the super family of proteins that includes receptors for all of the classical steroid hormones, such as estradiol, progesterone, testosterone, glucocorticoids, mineralocorticoids, thyroxine and retinoids. The nVDR protein contains a ligand binding domain able to bind with high affinity and with great specificity to 1α,25(OH)₂D₃ and closely related analogs.

Additionally, 1α,25(OH)₂D₃ has been found to generate biological responses via interaction with a putative membrane receptor, mVDR, which is coupled to cellular signal transduction pathways presently described as the “non-genomic” biological response. This interaction generates a rapid response via opening voltage gated Ca²⁺ channels and Cl⁻ channels as well as activating MAP-kinases. The response of the mVDR to its several ligands occurs rapidly, often producing indicia of binding within 5-10 minutes.

### Transverse Tubules

The transverse tubules (t-tubules) of mammalian cardiac ventricular myocytes (herein also referred to as ventricular cardiomyocytes) are invaginations of the sarcolemma and glycocalyx, which appears to remain associated with the sarcolemma within the t-tubules. Many of the proteins involved in excitation-contraction coupling appear to be concentrated at the t-tubules. Therefore, it has been suggested that the t-tubules play a central role in cell activation and muscle contraction, particularly in the cardiomyocyte

Consideration of the electrical properties of the t-tubules is important because it seems likely that the t-tubules are the most important site for excitation-contraction coupling in the cardiomyocyte. The local control theory of Ca²⁺ release is that local Ca²⁺ entry across the cell membrane, predominantly via I_{Ca}, triggers local Ca²⁺ release from an adjacent cluster of RyRs. The whole-cell Ca²⁺ transient is the temporal and spatial sum of these individual localized release events. Ca²⁺ sparks occur predominantly near the t-tubules, and localized Ca²⁺ release (Ca²⁺ spikes) occurs at discrete sites at the Z line. These spikes are proportional to I_{Ca} and to derived SR Ca²⁺ flux, providing strong support for the idea that local Ca²⁺ entry across the t-tubule membrane triggers local Ca²⁺ release from adjacent RyRs. Previous studies have shown that the expression of amphisin-2, a protein known for linking the plasma membrane and submembranous cytosolic scaffolds in CHO cells, can generate narrow tubules that are continuous with the plasma membrane. The t-tubule membrane appears to have a distinct protein and lipid composition and is enriched in cholesterol, which can be used as a tool to separate t-tubule and surface membranes that are useful for studying the receptor-ligand coupling important in cardiomyocyte contraction and relaxation.

Despite the evidence that the t-tubules are important in excitation-contraction coupling, there have been relatively few studies of this network during pathological conditions. The present disclosure describes assays that are in some embodiments, based on the finding that a previously undescribed mVDR located in or adjacent to the t-tubules (herein referred to as VDRₜₜ) exerts a direct and rapid contractile response of the cardiomyocyte in the presence of Vitamin D or Vitamin D compound. Without wishing to be bound by theory, this receptor may be a likely candidate for the observed improvement in contractile function of heart cells experiencing cardiomyopathy and hypertrophy due to the direct and acute phosphorylation of Ca²⁺ cycling and monofilament proteins implicated in cardiac myocyte contraction. Green, J et al., J. Mol. & Cell. Cardiol. (2006), 41:350-359.

The VDRₜₜ imbedded in plasma membrane and t-tubules of cardiomyocytes can be utilized to screen novel test compounds that bind specifically to the VDRₜₜ, or modulate the activity of VDRₜₜ. In some embodiments, screening methods can be adapted to screen test compounds to identify candidate heart failure compounds in a high throughput fashion.

Screening Assays for Agonists/Antagonists of (VDRₜₜ) Localized in the t-Tubules and Plasma Membrane

In some embodiments, the present disclosure provides screening assays involving a novel receptor located in and proximate to cardiomyocyte t-tubules. Preferably, the novel receptor is isolated within and/or adjacent to t-tubule invaginations. Accordingly, VDR localized in or adjacent to the t-tubules that are the subject of the present disclosure, are herein called t-tubule VDR (VDRₜₜ) to distinguish from the nuclear form of the VDR described above. Without being bound to any one particular theory, it is believed that the VDRₜₜ is intimately involved in the pathogenesis of cardiac hypertophy and heart failure and forms the basis of analytical and diagnostic screening assays for test compounds that bind and activate signaling by the VDRₜₜ and thereby the downstream processes resulting from the signaling from this receptor in cardiomyocytes.

In some embodiments, the present disclosure provide a method of screening for one or more test compounds that specifically bind to cardiomyocyte VDRₜₜ, and thereby affect the function of VDRₜₜ signaling. The screening method generally comprises a) contacting a cell, cell membrane or artificial construct having a functional VDRₜₜ imbedded in a membrane with one or more vitamin D analogs; and b) determining whether the compound specifically binds to the one or more VDRₜₜ by measuring for example, the binding kinetics of the vitamin D analog alone and in the presence of a known competitor, the level of signaling outputted by the receptor by measuring the level and degree of signal transduction, Ca²⁺ mobilization, by direct or indirect immunofluorescence or any commonly known mechanism of receptor binding transduction.

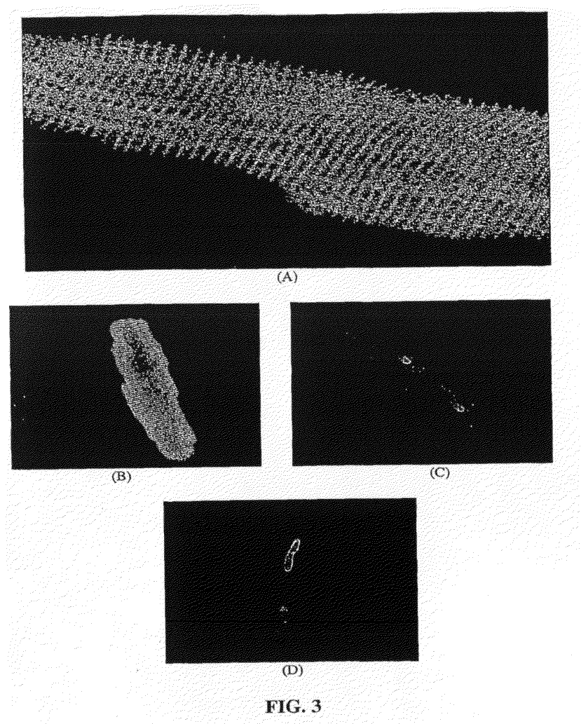

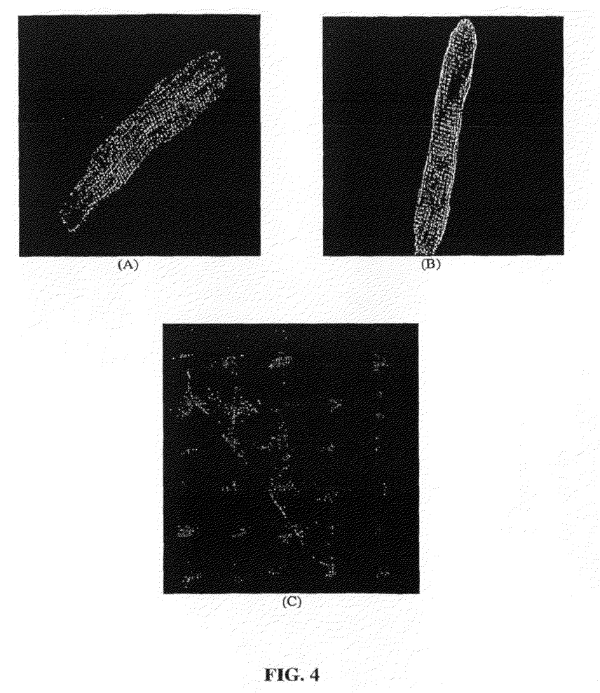

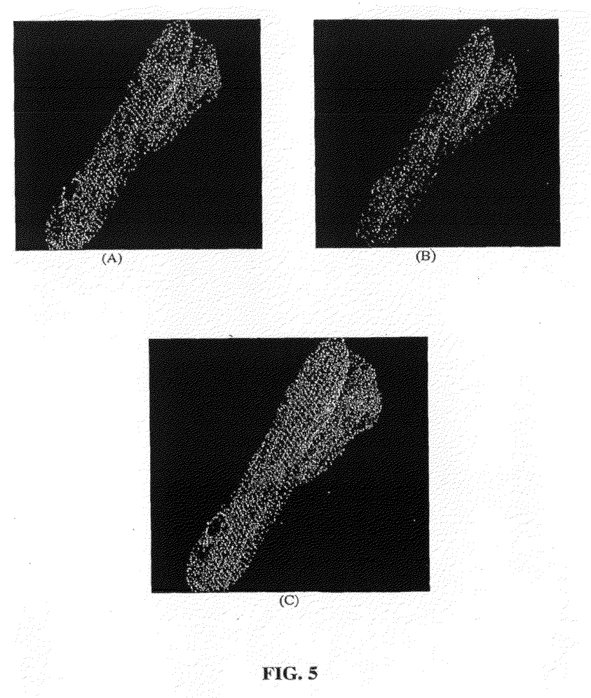

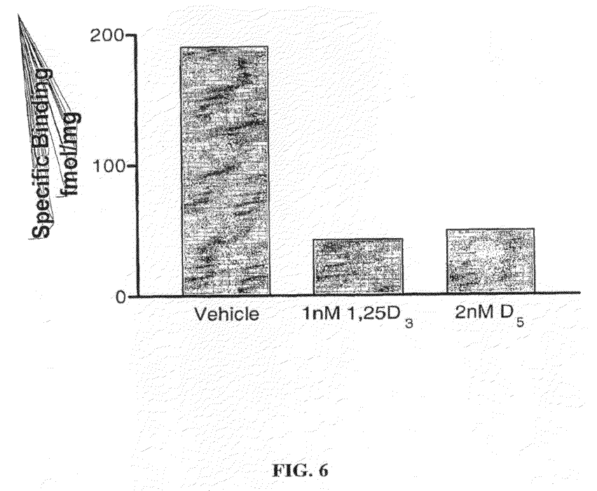

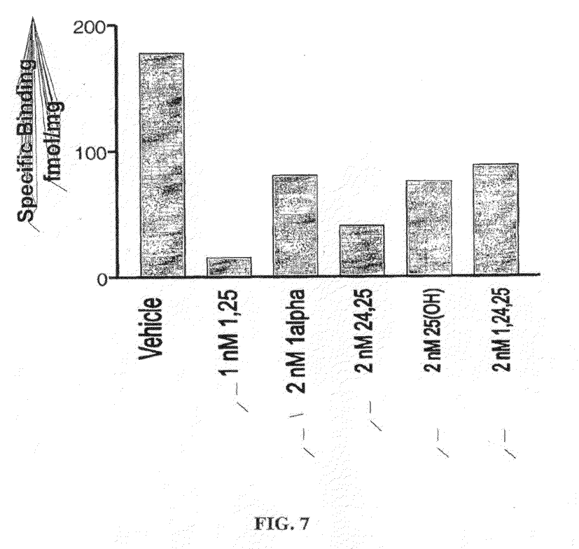

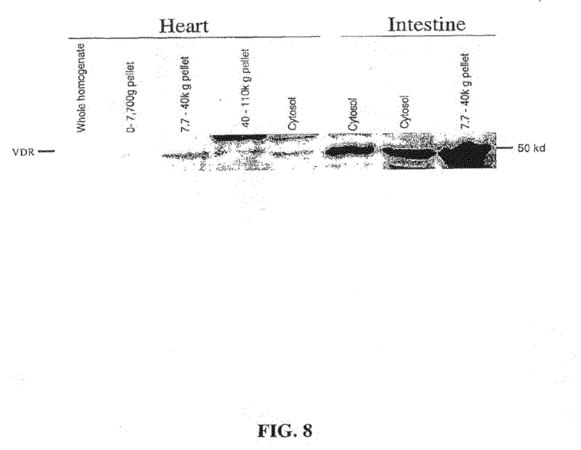

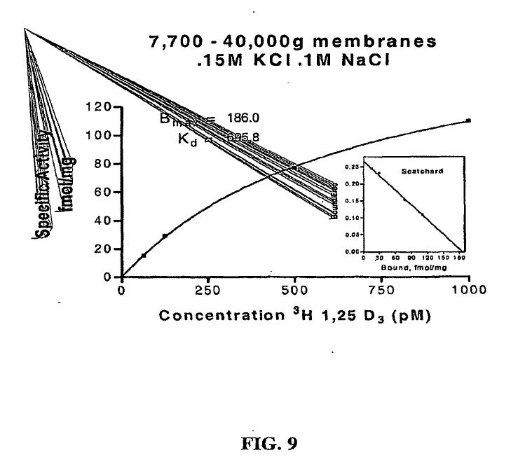

The putative VDRₜₜ has been identified and biochemically characterized during a search for membrane bound VDR isoforms. The deduced VDRₜₜ has an approximate molecular weight of 50 kDa. and can be identified by specific binding with antibodies directed against VDR (See FIGS. 3A-3D) including (sc1008 (anti-VDR C₂0)), sc13133 (monoclonal anti-VDR D₆) all commercially available from Santa Cruz Biotechnology Inc., Santa Cruz Calif., USA). The putative VDRₜₜ co-localizes in the t-tubules along with Serca 2 as evidenced by immunofluorescence studies of rat cardiomyocytes, as shown in FIGS. 4-5. Further biochemical characterization by radioligand binding experiments described herein show that this receptor has a one-site binding kinetics with 1α,25-dihydroxyvitamin D₃ and also binds with various affinities to vitamin D analogs including 1α,dihydroxyvitamin D₃; 24,25 dihydroxyvitamin D₃; 25-hydroxyvitamin D₃; 1,24,25 trihydroxyvitamin D₃ and Vitamin D₅. (as shown in FIGS. 6 and 7). The dissociation constant for VDRₜₜ obtained from rat cardiac myocyte membrane preparations is approximately 0.7 nM. The VDRₜₜ receptor Bmax was calculated at 186 fmol/mg protein as shown by Scatchard transformation in FIG. 8.

### Sources of VDRₜₜ

In some embodiments, methods include the steps of providing the VDRₜₜ either as soluble membrane extracts, transfected cell lines, isolated cardiomyocytes or liposomes having an embedded VDRₜₜ linked to a reporter, exposing the VDRₜₜ to the test compound under conditions that permit direct interaction between the VDRₜₜ and the test compound, and determining whether the test compound has altered the activity of the VDRₜₜ in comparison to a characterized vitamin D analog.

VDRₜₜ can be isolated from any mammalian muscle cell. In various embodiments, VDRₜₜ for screening purposes are isolated as membrane preparations from live heart tissue or from mature or neonatal cardiomyocytes that have been made immortal using genetic techniques for example HL-1 cells. Live heart tissue can be obtained from any experimental animal commonly used for the purpose of obtaining cardiomyocytes, including without limitation, from primates, mice, rats, dogs, and other laboratory animals commonly housed in any biomedical or pharmaceutical research facility. Cells that are functional cardiomyocytes obtained from animal hearts that have not been genetically altered are called wild-type cardiomyocytes.

Cardiomyocytes from live experimental animals can be isolated using any method commonly known in the art for myocyte isolation. The plasma membranes can be isolated using differential centrifugation techniques that is capable of subcelluar fractionation, or any commonly known method for isolating plasma membranes from myocytes, for example, L. Dombrowski et al., “A new procedure for the isolation of plasma membranes, T tubules, and internal membranes from skeletal muscle”, Am. J. Physiol. Endocrinol. Metab., (1996) Vol. 270(4):E667-E676, which is incorporated herein in its entirety.

In some embodiments, the cell lines can be derived from embryonic and/or non-embryonic cardiac myocytes transfected with oncogenes including, but not limited to c-myc (cellular myc), rat sarcoma (Ras) and Simian Virus 40 (SV40) large T antigen under the control of any cardiac cell gene promoter known in the art, for example α-á-cardiac MHC promoter. (Borisov, A. B. et al., (1995), Ann. N.Y. Acad. Sci., 752:80-91). In certain embodiments, the cell lines can express several detectable adult cardiac myocyte markers including, but not limited to, aα-MHC and aα-cardiac actin, yet other cardiac myocyte cell types can be included when studies relating to the development and dysregulation of immature cardiac myocytes (embryonic and neonatal myocytes for example) are performed including immature myocytes expressing b-MHC and act-skeletal actin.

In some embodiments, crude membrane preparations comprising t-tubules and plasma membranes from cardiomyocytes can be isolated and incubated with one or more test compounds. VDRₜₜ was found to be present in the 7,700-40,000×g membrane fraction of rat cardiomyocytes and only slightly present in the 40,000-110,000×g fraction as shown in FIG. 9 by Western blot analysis using the methods described herein in Example 4 below.

In some embodiments, the VDRₜₜ can also be expressed in cultured cells, for example, cell lines can be derived from embryonic and/or non-embryonic cardiac myocytes transfected with oncogenes including, but not limited to c-myc (cellular myc), rat sarcoma (Ras) and Simian Virus 40 (SV40) large T antigen under the control of any cardiac cell gene promoter known in the art, for example α-cardiac MHC promoter. (Borisov, A. B. et al., (1995), Ann. N.Y. Acad. Sci., 752:80-91). In certain embodiments, the cell lines can express several detectable adult cardiac myocyte markers including, but not limited to, α-MHC and α-cardiac actin, yet other cardiac myocyte cell types can be included when studies relating to the development and dysregulation of immature cardiac myocytes (embryonic and neonatal myocytes for example) are performed including immature myocytes expressing b-MHC and α-skeletal actin. In some embodiments, the cultured cardiomyocyte cell line is the murine atrial myocyte cell line HL-1 (herein HL-1 cells), capable of being passaged indefinitely.

In some embodiments, assays can be practiced with VDRₜₜ expressed in or adjacent to the plasma membrane and/or t-tubules from a variety eukaryotic cell samples, including viable cells, which can be, for example, transiently or stably transfected cells; whole cell lysates; or fractionated cell lysates. Several types of eukaryotic cells can be useful in the methods of the present disclosure, including primary and immortalized cells, and a variety of cell types such as cardiomyocytes, myocytes, fibroblasts and adipocytes. A eukaryotic cell sample also can be prepared from a tumor cell, for example, a melanoma, colon tumor, breast tumor, prostate tumor, glioblastoma, renal carcinoma, neuroblastoma, lung cancer, bladder carcinoma, plasmacytoma or lymphoma cell. In some embodiments, VDRₜₜ can be expressed in the plasma membrane of cultured cells. In some embodiments, convenient immortalized cell types are, for example, the human embryonic kidney cell line HEK293, the human cell line HeLa and the green monkey cell line CV-1. In some embodiments, stem cells can be transfected with a VDR nucleotide sequence derived from known animal VDR encoding nucleic sequences, including human and conditioned to differentiate into myocyte cell lineage using appropriate differentiation factors and other growth factors known in the art. Signal sequences for expression on the plasma membrane as opposed to the cytosol is also known in the art.

A eukaryotic cell sample useful in the invention can be prepared from transiently or stably transfected cells, or from an animal expressing an exogenous nuclear hormone receptor. Methods for stably or transiently introducing a vector or nucleic acid molecule into a eukaryotic cell are well known in the art and include calcium phosphate transfection, electroporation, microinjection, DEAE-dextran and lipofection methods (see, for example, Ausubel et al., Current Protocols in Molecular Biology, John Wiley & Sons, Inc., New York (2000)). A viral vector also can be useful to express an exogenous nuclear hormone receptor in a eukaryotic cell. Such a viral vector can be, for example, a retroviral vector, adenoviral vector, Herpes simplex virus vector, vaccinia virus vector, cytomegalovirus vector, Moloney murine leukemia virus vector, lentivirus vector, adeno-associated virus vector, or the like.

In some embodiments, VDRₜₜ is produced recombinantly using the nucleic acid sequence of VDRₜₜ encoding a membrane form of VDR having an approximate molecular weight of 50 kd, a binding dissociation constant Kd of about 0.7 nM, and a Bmax of between 100 and 500 fmol/mg protein. Any cell or artificial cell-free system expressing the VDR can be used in the screening methods described herein. In some embodiments, the source of VDR can be from any cell expressing a functional nuclear VDR or VDRₜₜ. In some embodiments, a method for screening for a candidate compound that modulates the activity of a Vitamin D receptor for the treatment of heart failure, the method comprises (a) contacting a cell having a Vitamin D receptor with a first sample comprising a test compound, thereby forming a treated cell, and thereafter observing one or more phenotypic parameters thereof selected from the group consisting of cell size, cell proliferation, and cell morphology and combinations thereof; (b) contacting under the same conditions as used in (a), a cell having a Vitamin D receptor with a second sample identical in composition to the first sample minus the test compound, thereby forming a control cell, and thereafter observing one or more phenotypic parameters thereof selected from the group consisting of cell size, cell proliferation, and cell morphology and combinations thereof; and (c) comparing the observed phenotypic parameters to find a significant difference between said treated and control cells; and (d) verifying that the test compound is a ligand for the Vitamin D receptor; and (e) identifying, based on a significant difference found in (c), the test compound as a candidate compound that modulates the activity of the Vitamin D receptor for the treatment of heart failure.

Thus, a cell that expresses a functional VDRₜₜ can be used, as long as VDRₜₜ activity is linked to some means for detecting a change in VDRₜₜ activity. In some embodiments, recombinant eukaryotic cells expressing mammalian VDRₜₜ or nVDR can be used. By recombinant cell, it is meant a cell that expresses, either transiently or stably, at least one nucleic acid sequence that has been introduced into the cell through human-directed activities. Thus, recombinant cells include cells that have been engineered through recombinant nucleic acid technology and through genetic recombination, as well as those created using other laboratory techniques known to those of skill in the art to express a functional VDRₜₜ or nVDR. In some embodiments, recombinant cells expressing functional VDRₜₜ or nVDR that are suitable for high throughput screening are used. In some embodiments, recombinant cells can comprise host cells that have a functional myocyte phenotype, or stem cells that can be differentiated into the myocyte and preferably the cardiomyocyte lineage. Specific methods of cloning functional VDR into cells can be found in Santiso-Mare, D. et al., Mol. Endocrinol. (1993) 7:833-839.

### Test Compounds for Screening Assays

In some embodiments, the test compound can be any chemical entity. Such an entity can be any chemical, salt or solvate thereof, for example, an organic molecule including: carbohydrate, steroid, polypeptide; small molecules; natural products; library extracts; and bodily fluids. In some embodiments, organic molecules having a generalized structure comprising seco-steroids and their metabolites and analogs. In some embodiments, seco-steroids including ergocalciferol or vitamin D₂ and cholecalciferol or vitamin D₃ are non-limiting examples of Vitamin D compounds. In some embodiments the test compound can include the following vitamin D analogs and their variants: 1α-hydroxyvitamin D₃; 25-hydroxyvitamin D₃; 1,24,25-(OH)₃D₃; 24,25-(OH)₂D₃; 1,25,26-(OH)₃D₃; 24,25-(OH)₂D₃; synthetic analogs of D2 and D3, including but not limited to 14-epi-19-nor, 20(R) and (S), hydroxypregnacalciferol, 1,25-dihydroxy-16-ene-23-yne-26, 27-hexafluorocholecalciferol; 25,26-dehydro-1α,24R-dihydroxycholecalciferol and 25,26-dehydro-1α,24S-dihydroxycholecalciferol; 1α-hydroxy-19-nor-vitamin D analogs; 26,28-methylene-1α,25-dihydroxyvitamin D₂ compounds; 1α-hydroxy-22-iodinated vitamin D₃ compounds; 23-Oxa-derivatives of vitamin D; and fluorinated vitamin D analogs; 20-methyl-substituted vitamin D derivatives; (E)-20(22)-Dehydrovitamin D compounds; 19-nor-Vitamin D₃ compounds with substituents at the 2-position; and 22-thio vitamin D derivatives. In some embodiments, the test compounds can also include Vitamin D₅ compounds (sitocalciferol), salts thereof, metabolites and analogs thereof. Test compounds can be any steroid, including seco-steroids that can mediate their effects if any, through the VDR receptor including the VDRₜₜ and the nVDR.

Candidate compounds for heart failure are test compounds that are capable of binding, or activating or modulating the activity of a VDR receptor, including nVDR, mVDR for example, VDRₜₜ receptor or can induce a cell, for example a cardiomyocyte, to modulate expression of a hypertrophy biomarker through a VDR. As used herein, a cardiac hypertrophy biomarker is any gene or gene product that is modulated, i.e. up-regulated or down-regulated by the disease condition and which is mediated through the VDR. For example, certain forms of PKC is down-regulated when the cardiomyocyte is hypertrophic. The decreased phosphorylation state of the cell may lead to suppression of other important factors that are necessary for proper contraction, growth, differentiation, and other cellular functions that are resulting in a state of hypertrophy. Alternatively, other cell factors or factors that influence the expression of genes and their products that are needed for proper cardiomyocyte function may be increased due to the hypertrophic state. In this case the biomarker is an increased expression of such a factor. In some embodiments, c-myc is increased during cardiac hypertrophy, upon addition of Vitamin D3, the cell decreases the level of c-myc which is over expressed when the cardiomyocyte is in a hypertrophic state. O′Connell, T., et al. “1,25-Dihydroxyvitamin D3 regulation of myocardial growth and c-myc levels in the rat heart.” Biochem. Biophys. Res. Commun. 213(1):59-65 (1995). In some embodiments, cardiac hypertrophy biomarkers can include: c-myc, myotrophin, PKC, phospholamban phosphorylation, ANP, calbindin 9, intracellular Ca2+ levels, phosphorylation of cardiac Troponin I, and any other gene or gene product that is specifically up-regulated or specifically down regulated resulting in the cardiac hypertrophy or as a result of cardiac hypertrophy.

In some embodiments a method for screening for a candidate compound that modulates the activity of a Vitamin D receptor for the treatment of heart failure is provided. The method comprises (a) contacting a cell having a vitamin D receptor and expressing at least one biomarker for cardiac hypertrophy with a test compound in a diluent, to form a test cell. (b) contacting the same cell type as in step (a) with the diluent in the absence of the test compound, to form a control cell. (c) verifying that the test compound is a ligand for the Vitamin D receptor. (d) comparing the level of expression of the biomarker in the test and control cells to identify a significant difference therein and (e) identifying based on a significant difference found in (d), the test compound as a candidate heart failure compound.

As discussed above, in some cases the biomarker is typically over-expressed in the hypertrophic disease state and the candidate heart failure compound reduces the expression of this biomarker. In other cases, the biomarker is repressed or reduced in magnitude when evaluating the biomarker in the hypertrophic disease state, and a candidate heart failure compound increases, or elevates or restores the biomarker to a level that is commensurate with healthy cells or cardiomyocytes. For example, a test compound that when bound to a VDR can increase the levels of myotrophin, or PKC activation and expression, the test compound is said to be a candidate heart failure compound. Alternatively, if a test compound is found to bind to the VDR and reduce the cellular levels and/or expression of c-myc, ANP, calbindin 9 in the cell or cardiomyocyte, then the test compound is a candidate heart failure compound.

In some embodiments, the test compounds can be used in any form, for example, a purified form, a partially purified form, as the sole solute in a solution, as one of two or more solutes in a solution as a component in a complex mixture or solids, liquids and or gases.

### Binding Assays

The screening assays described in the present disclosure can be used to identify test compounds that have the capability to specifically bind to the VDRₜₜ in cell-based, or cell free assays. A VDR agonist is generally any agent that can bind, cross-link or ligate a VDR including nVDR and VDRₜₜ and stimulate the activity of the VDR through downstream signaling or receptor activation. For example, stimulating the VDRₜₜ receptor can be determined by the ensuing downstream signal transduction for example PKC activation and phosphorylation of another factor, protein or signaling molecule. In some embodiments, the screening assays can utilize a cell based or cell free binding assay having VDRₜₜ and at least one test compound present. In some embodiments, a method of screening for test compounds that are heart failure candidate compounds, wherein the method comprises: (a) contacting a test compound with VDRₜₜ in a reaction mixture, wherein the reaction mixture conditions permits the test compound and a labeled binding partner to bind to the VDRₜₜ binding site and form a labeled binding complex; (b) determining the level of labeled binding complex in the presence of the test compound; and (c) comparing the level of the labeled binding complex in the presence of the test compound and labeled binding partner to the level of labeled binding complex in the presence of the labeled binding partner and in the absence of the test compound, wherein an decreased formation of labeled binding complex in the presence of the test compound indicates that the test compound is a heart failure candidate compound.

In some embodiments, the screening assays can comprise a radiolabeled binding assay. The radio labeled binding assay and variations thereof, can be designed using membrane preparations of VDRₜₜ and thereby incubating the VDRₜₜ with one or more test compounds in an assay mixture and for sufficient time for the test compound to specifically bind to the VDRₜₜ. In some embodiments, a radiolabeled competitor (an innate binding partner) which is known to specifically bind to the binding domain of the VDR is added. If the test compound can displace any amount of the radiolabeled binding partner, then the test compound is said to specifically bind to the VDRₜₜ, and is a candidate compound for further screening. In various embodiments, the innate binding partner can be labeled with any molecule, for example, radioisotope, fluorescent dyes, enzymatic reporters such as alkaline phosphatase, or horseradish peroxidase, biotin, avidin, enzyme or detection molecule, for example, HIS tag, FLAG tag and the like, to determine whether the test compound can specifically bind to VDRₜₜ.

In some embodiments, test compounds can be compared to the specific binding of known Vitamin D analogs, including 1,25 dihydroxy D₃, 1α,dihydroxy D₃, 24,25 dihydroxy D₃, and 1, 24, 25 dihydroxy D₃ and Vitamin D₅. The specific binding results of these known Vitamin D analogs can be useful as an indicator of the clinical relevance of an unknown test compound when compared to a known Vitamin D in screening assays employing VDRₜₜ derived from animal cardiomyocytes, for example as shown in FIG. 7 using VDRtt derived from membrane preparations from rat cardiomyocytes using radiolabelled 1,25 dihydroxy Vitamin D₃ as a competitor.

In some embodiments, binding assays involving competitor ELISA assays can be used to screen for candidate heart failure compounds. This employs a sandwich assay wherein the innate binding partner, for example 1,25(OH)₂D₃ is coated on the bottom of ELISA plates comprising 96, 144, or any commercially available multiwell format. Solubilized VDRₜₜ or membrane preparations comprising VDRₜₜ can then be added to the wells of the plates coated with 1,25(OH)₂D₃. After a washing step, the test compound can be added at one or more concentrations to the wells containing the innate binding partner and VDRₜₜ. The mixture can be incubated for a sufficient period of time to allow the competition between the innate binding partner and the test compound for the VDRₜₜ to reach equilibrium. The mixture can then removed from the wells using a gentle wash and any residual VDRₜₜ is measured using an antibody directed to VDR, for example, sc1008 (anti-VDRₜₜ C₂0), sc13133 (monoclonal anti-VDR D₆), both from Santa Cruz Biotechnology Inc., CA, USA. If the presence of antibody labeled VDRₜₜ is lower in the assay wells containing the innate binding partner and test compound as compared to the innate binding partner in the absence of the test compound, then the test compound is a candidate heart failure compound. Alterations to these assay designs combining the binding of a test compound to VDRₜₜ in the presence of a known binding molecule such as 1,25(OH)₂D₃ is relatively well known. The embodiments described herein further encompass such modifications.

In some embodiments, VDRₜₜ can be bound to the surface of a substrate and then incubated with labeled innate binding partner 1,25(OH)₂D₃ which can include any radiolabel for example [³H] or fluorescence labeled 1,25(OH)₂D₃. Displacement of the labeled innate binding partner from the VDRₜₜ receptor with a test compound indicates that the test compound is a candidate compound for the treatment of heart failure.

In some embodiments, assays contemplated by the present can be designed to screen for test compounds which can bind and modulate the activity of a VDR including, nVDR and VDRₜₜ with its cognate secondary messengers and signal transduction partners, for example PKC. It is known that specific binding of 1,25(OH)₂D₃ in the presence of VDR results in rapid, non-genomic, PKC-mediated phosphorylation of Ca²⁺ cyling proteins and myofilament proteins. Green, J J. et al., J. Mot. & Cell. Cardiol. (2006) 41:350-359. In some embodiments of the present disclosure, cell based and cell free assays can be used to screen for candidate compounds that specifically bind and modulate the activity of VDR including nVDR and VDRₜₜ and modulate the ability of the VDR to activate or repress a cardiac biomarker.

In some embodiments, eukaryotic cells expressing functional VDRₜₜ can be incubated with a test compound and in a separate reaction with a known binding partner such as 1,25(OH)₂D₃. The samples are treated under the same assay conditions. The degree of VDRₜₜ activation in the presence of the test compound and of the innate binding partner can be determined by measuring the rapid response activity of the VDR by measuring PKC phosphorylation by incubating the cells with radiolabelled ATP and measuring the radioactivity of the cells due to PKC phosphorylation with and without the test compound. Test compounds that can modulate the activity of PKC or any other cardiac hypertrophy biomarker via interaction and specific binding with the VDR, including nVDR and VDRₜₜ as compared to a control solution having no test compound are candidates for heart failure compounds.

In some embodiments, VDRₜₜ activation and modulation can also be assayed using test compounds in a cell free system. In some embodiments of the present disclosure screening methods can comprise the steps of providing membrane preparations comprising VDRₜₜ isolated from cells as described above. The membranes can contain a functional VDR and can be placed into assay reactions comprising PKC, secondary messengers and transcriptional regulators of calcium cycling proteins, for example SERCA 2. SERCA 2 is intimately involved in Ca²⁺ cycling (via phosphorylated phospholamban) and is a key regulator of cardiomyocyte contraction and relaxation. Assays designed to measure PKC activity are well known in the art. For example, Slater, S J. et al., J. Biol. Chem. (1995), 270(12):6639-6643 and Slater, S. J., et al., (1994), J. Biol. Chem. 269:17160-17165. PKC activity can be measured by measuring the phosphorylation of phospholamban or some other PKC specific substrate having a phosphorylation amino acid residue (serine or threonine). Phospholamban phosphorylation can be measured using a specific antibody to phospholamban (Phospholamban antibody [2D12] (ab2865) (AbCam Inc., 1 Kendall Square, Step 341 Cambridge, Mass., USA), isolated membranes containing VDRₜₜ, radioactive γ³²P, ATP and other cell constituents, buffers and materials. PKC activity can be assayed using commercial kits provided, for example by Promega (SignaTECT® Protein Kinase C (PKC) Assay System, Promega Corp., Madison, Wis., USA). Test compounds that can increase or decrease the level of phosphorylation due to PKC activity as a result of VDRₜₜ modulation when the VDRₜₜ is in contacts with a test compound as compared to calcitriol or 1,25(OH)₂D₃ can be candidate compounds for heart failure.

In some embodiments, a method of screening for test compounds that are heart failure candidate compounds, the method comprising: contacting a test compound to a reaction mixture, the reaction mixture comprising:

(i) a VDRₜₜ polypeptide derived from a cardiomyocyte,

(ii) a Protein Kinase C polypeptide

(iii) a cell extract comprising radiolabeled ATP and a PKC specific substrate

wherein the reaction mixture conditions permit binding of the VDRₜₜ to the cell extract containing Protein Kinase C to phosphorylate the PKC specific substrate and after a suitable incubation period, detecting levels of formation of the phosphorylated PKC specific substrate in the reaction mixture in the presence of the test compound; and measuring the amount of Protein Kinase C activity in the presence of the test compound and of a control, wherein an increase in the amount of Protein Kinase C activity in the presence of the test compound as compared to the control indicates that the test compound is a heart failure candidate compound.

In some embodiments, the specific PKC substrate is phospholamban, or any commercially available PKC specific substrates, for example, H-Arg-Arg-Gly-Arg-Thr-Gly-Arg-Gly-Arg-Arg-Gly-Ile-Phe-Arg-OH among others provided (Calbiochem, San Diego, Calif., USA).

### Assays Measuring Direct Effects on Cell Morphology

In some embodiments, cultured stable cells are used in screening assays to determine whether a test compound has an effect on a cardiomyocyte biomarker implicated in cardiac hypertrophy. The stable cell lines used in the screening assays can express differentiated myocyte phenotype including, but not limited to, cytoplasmic reorganization and myofibrillogenesis similar to that observed in mitotic cardiomyocytes of the developing heart, the presence of highly ordered myofibrils and cardiac-specific junctions, the ability to undergo spontaneous contractions similar to in vivo immature mitotic cardiomyocytes, expression of ANP, α-cardiac actin, desmin, myotrophin, and calbindin-9, and the presence of several voltage-dependent currents that are characteristic of a cardiac myocyte phenotype not commonly found in non-cardiac cells.

In some embodiments, the cell lines can be derived from embryonic and/or non-embryonic cardiac myocytes transfected with oncogenes including, but not limited to c-myc (cellular myc), rat sarcoma (Ras) and Simian Virus 40 (SV40) Large T antigen under the control of any cardiac cell gene promoter known in the art, for example α-cardiac MHC promoter. (Borisov, A. B. et al., (1995), Ann. N.Y. Acad. Sci., 752:80-91). In certain embodiments, the cell lines can express several detectable adult cardiac myocyte markers including, but not Limited to, α-MHC and α-cardiac actin, yet other cardiac myocyte cell types can be included when studies relating to the development and dysregulation of immature cardiac myocytes (embryonic and neonatal myocytes for example) are performed including immature myocytes expressing MHC and α-skeletal actin.

In an illustrative example, a method for screening for compounds that inhibit heart failure comprises HL-1 cells grown or incubated in medium containing a test compound. The number, size, shape, and morphology of the cells are assayed. For example, cells can be fixed and stained and examined using light microscopy. Alternatively, cells can be fixed, stained, sectioned, and examined using electron microscopy. Alternatively, cells can be fractionated using density centrifugation. Cell cultures treated with a test compound that prevents HL-1 cells from increasing in number, size and reverting to an immature form compared to HL-1 cells grown or incubated under similar conditions but without the test compound indicates that the test compound inhibits heart failure.

Biomarkers of cardiac hypertrophy can include bioactive agents or factors that modulate the contractility of the cardiomyocyte, that regulate calcium homeostasis, that are implicated in remodeling the cardiomyocyte from a normal state to a hypertrophied state, and markers that are specifically down regulated in hypertrophied cells. Biomarkers can also include genes and their encoded proteins that are involved in cardiomyocyte energetics, muscle contraction ad signaling that can be modulated by Vitamin D compounds acting through the VDR. Such biomarkers can be identified by expression profiling obtained by screening whole genome libraries of amplified RNA obtained from hypertrophied heart biopsies representing a pool of heart failure patients in comparison to tissue samples taken from non-hypertrophied heart tissue. Methods identifying gene expression changes as a result of cardiac hypertrophy or cardiomyopathy are known in the art, for example, as described by Grezeskowial et al., “Expression profiling of human idiopathic dilated cardiomyopathy”, Cardiovascular Research, 59(2):400-411, (2003), which is incorporated by reference herein.

Murine atrial myocyte cell line HL-1 (herein HL-1 cells), are capable of being passaged indefinitely are grown in cell culture and incubated in medium containing a test inhibitor compound. In some embodiments, if the test compound prevents the HL-1 cells from proliferating and increasing in size, the compound can then be tested for its ability to regulate or modulate genes associated with cardiac myocyte proliferation and hypertrophy including PKC, c-myc, PCNA, ANP, calbindin 9 and 32, myotrophin, phospholamban, and SERCA2. In some embodiments, the test compound to be screened is compared to a control substance known to inhibit the action of proliferative genes including phorbol myristate acetate and endostatin. If the test compound inhibits the growth and proliferation of the HL-1 cells and can modulate one or more of the genes associated with cardiac hypertrophy, then the test compound is identified as candidate for a compound that inhibits or inhibits the progression of heart failure.

After a first time period, a test compound or control substance (vehicle) is added to the sample. After a second time period after said time point has elapsed, the cells are harvested for counting and other assays used in screening test compounds that inhibit or inhibit the progression of heart failure. For control assays (no test compound), HL-1 cells receive the test compound vehicle alone for example, 0.1% ethanol and incubated under similar conditions as cells incubated with the test compound. In some embodiments, the second time period can be 0 to 10 days, 1 to 5 days or 2 to 4 days.

At various time points, the HL-1 cell cultures incubated with the test compound and the vehicle and are subsequently washed and resuspended in media and removed from the cell culture dish and counted. In some embodiments, cells can be counted in any automated or non-automated cell counting/analysis device, including, but not limited to, coulter counters (Coulter Electronics, Hialeah, Fla.), flow cytometers, manual microscopic slide counts using hemocytometers and turbidity estimation using standard cell curves using a spectrophotometer at one or more wavelengths.

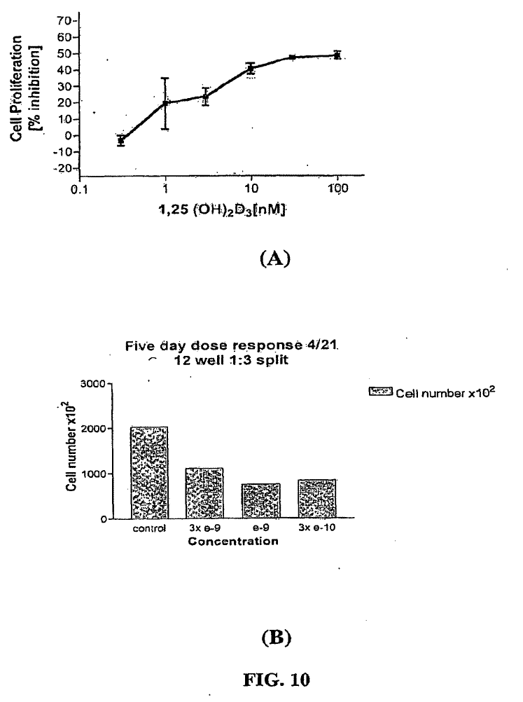

In certain embodiments, test compounds can be screened for their ability to inhibit proliferation of BL-1 cells in culture. As shown in FIG. 10 HL-1 cells incubated with a test compound for example, a vitamin D compound or a steroid, or small molecule, show a dose dependent inhibition of HL-1 growth in the presence of test compound Vit D when compared to similarly treated HL-1 cells incubated with a vehicle. A significant increase in the inhibition of growth of HL-1 cells treated with a test compound compared to the growth of HL-1 cells grown under similar conditions but without the test compound indicates that the test compound is a candidate for a compound that inhibits heart failure.

In some embodiments HL-1 cells can be assayed for the ability to grow (in size rather than in number) in the presence and absence of a test compound. As shown in FIG. 10(B), a range of concentrations of a test compound can be screened to identify the optimal concentration of the test compound to be used in inhibiting the proliferation of differentiated cardiac myocyte cells.

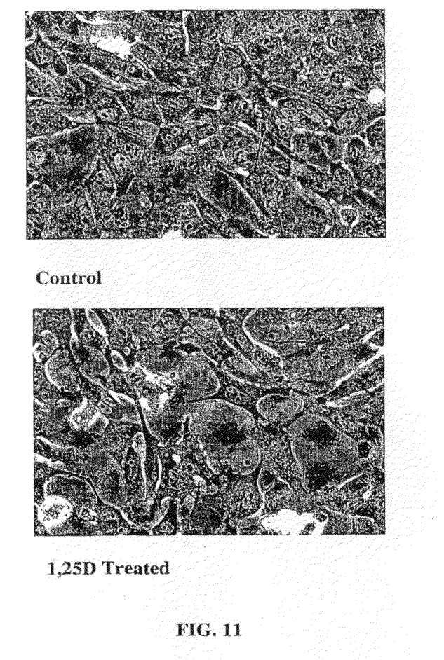

In some embodiments, screening assays can be included to determine whether the test compound can alter the structural morphology of the cardiac myocyte in cell culture. HL-1 cells can be incubated in the presence and absence of a test compound to determine whether structural changes can be observed by light microscopy. As shown in FIG. 11, HL-1 cells incubated with a test compound including, but not limited to, Vitamin D reveals the growth and presence of structural appendages called dendrites, which are morphological signs of maturation and differentiation of an adult myocyte phenotype. In contrast, the control HL-1 cells incubated with control vehicle alone, failed to show any morphological transition to the more mature form. A significant increase in dendrite formation in the HL-1 cells that were treated with a test compound compared to HL-1 cells grown under similar conditions without test compound indicates that the test compound is a candidate for a compound that inhibits heart failure.

In some embodiments, A method for screening for a candidate compound that modulates the activity of a Vitamin D receptor for the treatment of heart failure, the method comprising: (a) contacting a cell having a Vitamin D receptor with a first sample comprising a test compound, thereby forming a treated cell, and thereafter observing one or more phenotypic parameters thereof selected from the group consisting of cell size, cell proliferation, and cell morphology and combinations thereof; (b) contacting under the same conditions as used in (a), a cell having a Vitamin D receptor with a second sample identical in composition to the first sample minus the test compound, thereby forming a control cell, and thereafter observing one or more phenotypic parameters thereof selected from the group consisting of cell size, cell proliferation, and cell morphology and combinations thereof; (c) comparing the observed phenotypic parameters to find a significant difference between said treated and control cells; and (d) verifying that the test compound is a ligand for the Vitamin D receptor; and (e) identifying, based on a significant difference found in (c), the test compound as a candidate compound that modulates the activity of the Vitamin D receptor for the treatment of heart failure. With respect to cell size, proliferation and morphology changes, a candidate heart failure compound when incubated for a reasonable period, for example: 1 hour to 72 hours, 2 hours to 36 hours, 4 hours to 48 hours will increase the size of the cell or cardiomyocyte as compared to a control vitamin D3. With respect to proliferation, incubation for a reasonable period as set forth above, with a candidate heart failure compound will inhibit proliferation of the cell or cardiomyocyte. With respect to changes in morphology when a cell or cardiomyocyte is incubated with a candidate heart failure compound, the morphology is subjectively analyzed and will induce changes in morphology that resemble cells or cardiomyocytes having increased projections relative to a control without a test compound.

### Methods of Screening for Test Compounds Capable of Inhibiting Gene Products Known to Mediate Cardiac Hypertrophy in Cardiac Myocytes

In some embodiments, methods for screening serve to identify inhibitors of heart failure using assays that measure the ability of the test compound to inhibit gross structural changes that are often associated with reversion of the mature myocyte into the immature form. Such structural changes can include reversion to immature growth patterns, hyperplasia, and hypertrophy. In some embodiments, the present invention further provides methods for screening for candidate heart failure compounds using screening assays that detect the presence, and measures the expression of various biomarkers, including, Protein Kinase C isoforms, c-myc, calbindin 9 and 32, ANP, phospholamban, cardiac troponin I, and myotrophin. These factors have all been reported as implicated in the hypertrophied cardiac myocyte and are associated with Protein Kinase C enzyme activation via the VDR.

Test compounds that modulate the expression or activity of these biomarkers can modulate the biomarker in a reverse pattern to what is found in the hypertrophic state can be said to inhibit or reverse the progression of heart failure. In the following methods of screening, assays for determining intracellular c-myc, calbindin 9 and 32, ANP and myotrophin protein levels and/or locations are used. This can be done using any of the standard techniques of protein detection known in the art. The protein detection assays employed herein can be those described in Harlow and Lane (Harlow, E. and Lane, D., 1988, Antibodies: A Laboratory Manual, Cold Spring Harbor Laboratory Press, Cold Spring Harbor, N.Y.), which is incorporated herein by reference in its entirety.

These assays include, but are not limited to, immunological assays, including Western blots, immunohistochemistry, solid-phase radioimmunoassays, in situ hybridizations, and immunoprecipitations. Antibodies to c-myc, calbindin 9 and 32, ANP and myotrophin protein levels are known in the art, and can be commercially available or can be readily generated using well-known techniques. These same factors can be detected by detecting their cognate nucleic acid including DNA and mRNA. In some embodiments, these assays include, but are not limited to, in situ hybridization, Reverse Transcript-Polymerase Chain Reaction (RT-PCR), quantitative RT-PCR and Northern blotting.

The activation of Protein Kinase C in different cells results in a varied array of cellular responses, thus illustrating that Protein Kinase C plays an important role in many aspects of cell growth and metabolism. In some embodiments, screening assays are developed to address whether the test compound can increase Protein Kinase C phosphorylation of factors which regulate genes that are implicated in heart failure, including, but not limited to c-myc, ANP, VDR, calbindin 9 and 32 and myotrophin, and phosphorylation of factors directly such as phospholamban and cardiac troponin I. In some embodiments, qualitative and quantitative assays can be used to measure the expression of Protein Kinase C isoforms in VDR expressing cells, including cardiomyocytes and HL-1 myocytes. In some embodiments, biomarkers that are known to mediate the etiology and progression of heart failure, which are regulated by Protein Kinase C activity are also measured in screening assays of the present disclosure.

In a non-limiting method for screening for candidate compounds that inhibit heart failure, HL-1 cells are grown or incubated in medium containing a test compound. In certain embodiments the reference to which the test compound is compared can be a standard or control of any type, including average data generated in other assays, data generated in a control sample run concurrently with a given test. The presence, concentration, or amount of Protein Kinase C isoforms including biomarkers c-myc, VDR, calbindin 9 and 32 and myotrophin in the HL-1 cell is determined using a protein detection assays as described above. Test compounds that cause HL-1 cells to increase Protein Kinase C activity and increase phosphorylation of factors that down-regulate c-myc, ANP, calbindin 9 and 32 and up-regulation and/or expression of myotrophin than assays prepared in parallel comprising HL-1 cells grown or incubated under similar conditions but without the test compound are candidates for compounds that inhibit heart failure.

Other qualitative assays can be used, such as, e.g., microscopic examination of cells treated with the test compound. For example, cell staining techniques, as known in the art, can be used. Cells can be grown or incubated in cell culture medium containing the presence or absence of a test compound. In some embodiments, the test compound can be a Vitamin D analog or metabolite of Vitamin D₂ or Vitamin D₃. The HL-1 cells are incubated with the test compound or control vehicle as described above. After a period of incubation, the HL-1 cells are stained using primary antibodies that can be chosen from those including but not limited to, anti-Protein Kinase C antibodies, anti-c-myc, anti-VDR, anti-calbindin 9 and 32, and anti-myotrophin primary antibodies.

After a finite period of incubation with the primary antibodies the HL-1 cells are then washed and incubated with secondary antibodies conjugated with a radiolabel, an enzyme matched for the species used in the primary antibody incubation step. After another period of incubation, the then examined microscopically. In a non-limiting example of a method of screening using this type of assay, HL-1 cells are grown or incubated in medium containing a test compound, and prepared for cell staining using techniques commonly known in the art. See, e.g., Harlow and Lane, 1988, supra.

The anti-Protein Kinase C antibodies, anti-c-myc, anti-VDR, anti-ANP, anti-calbindin 9 and 32, and anti-myotrophin primary antibodies can be conjugated to a moiety allowing for its detection. Preferably, a secondary antibody is used. The secondary antibody recognizes and binds to the primary antibody. Preferably, the secondary antibody is conjugated to a moiety allowing for its detection. Alternatively, a tertiary antibody can also be used. The tertiary antibody is preferably conjugated to a moiety allowing for its detection.

Examples of moieties allowing for the detection of antibodies include fluorescent molecules (for example, fluorescein, rhodamine, Hoechst 33258, or Texas red), enzymes (for example, horseradish peroxidase, alkaline phosphatase, or beta-galactosidase), gold particles, radioactive isotope, and biotin. An assay is selected based on the labeling moiety used. For example, fluorescence microscopy can be used to detect fluorescently labeled antibodies. For cells stained with enzyme-conjugated antibodies, the cells are further treated with an appropriate substrate for conversion by the antibody-bound enzyme, followed by examination by light microscopy. Gold-particle labeled antibodies can be detected using light or electron microscopy. Isotope-labeled antibodies can be detected using radiation-sensitive film.

For cells stained with biotin-conjugated antibodies, the cells are further treated with streptavidin or avidin. The streptavidin or avidin is conjugated to a moiety that allows for detection such as, for example, a fluorescent molecule, an enzyme, gold particles, or radioactive isotope. In some embodiments, the HL-1 cells are co-stained with an antibody or antibodies specific for particular subcellular compartments (e.g., nucleus, cytoplasm, endoplasmic reticulum, etc.). Using any one of these techniques, or any other known technique for detecting antibodies in antibody-stained cells, the subcellular distribution of protein factors that are implicated in the etiology and/or progression of heart failure can be determined. If the test compound causes a decreased amount in any one of Protein Kinase C isoforms, c-myc, ANP and calbindin 9 and 32 primary antibodies to be found in the nucleus or cytoplasm of HL-1 cells, then it inhibits heart failure.

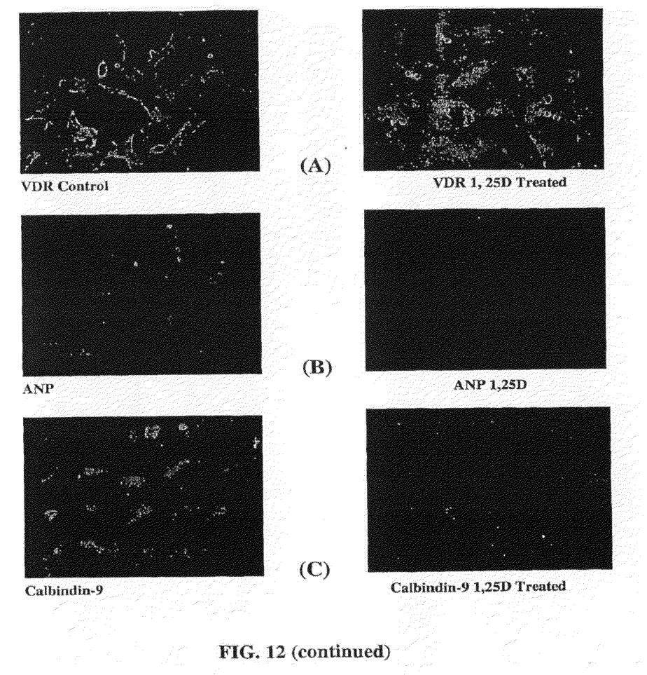

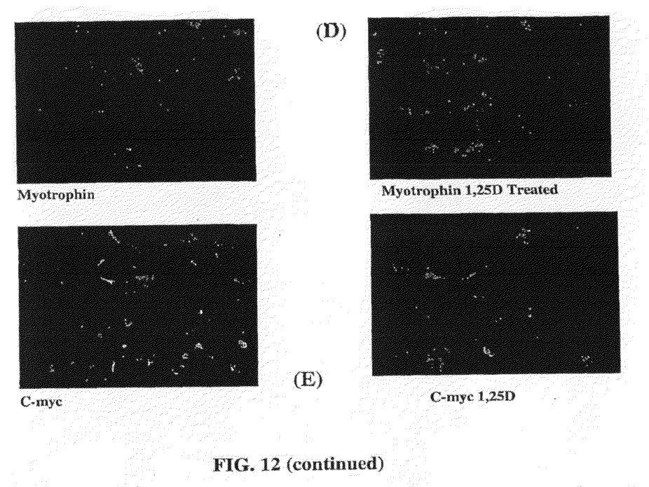

In certain embodiments, the test compound can be Vitamin D. As shown in FIG. 12, HL-1 cells treated with Vitamin D inhibited or reduced the expression of c-myc, calbindin 9 and ANP. The Vitamin D increased the expression of myotrophin and VDR in HL-1 cells.

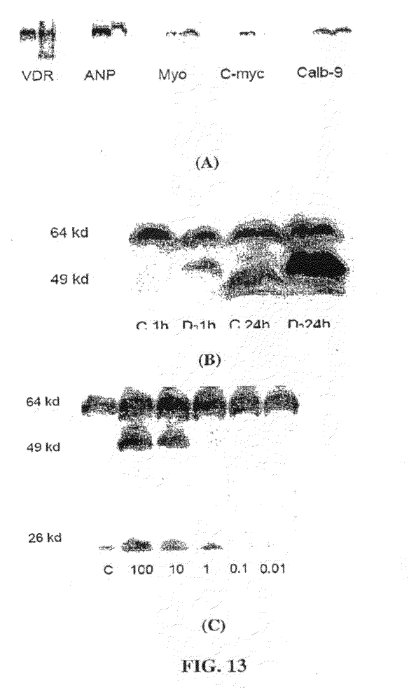

Screening assays employing incubation of VDR containing cells with and without a test compound. Cell extracts can be prepared and screened for increases or decreases of cardiac hypertrophy biomarkers or increase in PKC activity, VDR expression, myotrophin expression, using antibody detection methods described herein. Equivalent protein quantities of test compound cell extracts and control cell extracts can be loaded onto continuous or discontinuous PAGE gels and electrophoresed from 15 min to 24 hours, depending on the size of the gel. Following electrophoresis, the proteins can be electrophoretically transferred to PVDF membranes and probed with antibodies specific for example to Protein Kinase C and downstream regulated products c-myc, VDR, ANP, calbindin 9 and 32 and myotrophin. Bound antibodies can be visualized using chemiluminescent detection, chromatic detection or any method of detection of labeled membrane bound antibodies otherwise known as Western blotting. As shown in FIG. 13, HL-1 cells incubated in the presence Vitamin D₃ had significantly reduced levels of intracellular ANP, c-myc and Calbindin 9. In FIG. 13(A), Western blots reveal that when HL-1 cells are incubated in the presence of a control sample (left lane) and a test compound (right lane). In some embodiments, for example, Vitamin D₃ analog or metabolite, can be the test compound, or Vitamin D₃ can be a control to measure the activity of the test compound. As shown in FIG. 13(A), Vitamin D₃ reduces the expression of ANP, c-myc and calbindin 9 as compared to cells not incubated in the presence if Vitamin D₃. FIG. 13(B) shows that expression of the VDR is up-regulated when the HL-1 cells are incubated in the test compound Vitamin D₃. Similarly, FIG. 13(C) shows that the expression of the VDR is dose dependent when compared to the control. Vitamin D₃ has a positive up-regulating effect upon the expression of the VDR in HL-1 cells. FIG. 13(A) reveals a significant decrease in expression of ANP, c-myc and calbindin 9 in the HL-1 cells that were treated with a test compound compared to HL-1 cells grown under similar conditions without test compound indicating that the test compound Vitamin D3 as an example is a candidate for a compound that inhibits heart failure.

### Microarrays

The pace of cardiac hypertrophy drug research can be severely retarded when experimental assays require the use of patient/deceased heart tissue samples. In certain embodiments, the assays described herein can provide a convenient and inexpensive means to study and screen test compounds that affect the morphological, biochemical, and electrophysiological characteristics of myocytes having a heart failure phenotype via interaction with the non-genomic, and genomic form of VDR. The terms “array” and “microarray” are used interchangeably and refer generally to any ordered arrangement (e.g., on a surface or substrate) or different molecules, referred to herein as “probes”. Each different probe of an array specifically recognizes and/or binds to a particular molecule, which is referred to herein as its “target”. Microarrays are therefore useful for simultaneously detecting the presence or absence of a plurality of different target molecules, e.g., in a sample. The presence or absence of that probe's target molecule in a sample may therefore be readily determined by simply analyzing whether a target has bound to that particular location on the surface or substrate.

In some embodiments, the mircroarrays of the present disclosure comprise a solid non-porous substrate, such as glass slide or a silicon chip. In a typical microarray screening assay, the substrate is contacted with a sample containing biomaterials to be analyzed. The substrate is then contacted with probe molecules such as labeled nucleic acids or polypeptides or other molecules. The labeled molecules bind with the molecules in the sample. The unbound probe molecules are removed, for example, by washing, and the microarray is then read by a suitable signal detection device, for example, by fluorescence emission.

In some embodiments, the microarray comprises anchoring a protein, e.g., a VDR, nVDR or VDRₜₜ, onto a solid phase and detecting complexes of the protein and the test compound that are on the solid phase at the end of the reaction and after removing (e.g., by washing) unbound ligands for example unbound test compound and labeled Vitamin D control binding partner, for example, 1,25(OH)₂D₃. In some embodiments of such a method, a VDRₜₜ may be anchored onto a solid surface and a test compound is added with or without a labeled 1,25(OH)₂D₃ ligand. After incubating the test compound for a sufficient time and under sufficient conditions that a complex may form between the VDR protein and the test compound. Unbound test compound and unbound labeled 1,25(OH)₂D₃ are removed from the surface (e.g., by washing) and labeled molecules which remain are detected and measured. Test compounds that decrease the number of labeled 1,25(OH)₂D₃ actively bound in the complex are candidate heart failure compounds.

In some embodiments, one or more different test compounds are attached to the solid phase and then contacted with a functional VDR for example, nVDR or VDRₜₜ either in a soluble form, or in a membrane embedded form, and incubated under conditions which allow specific binding between the VDR and the test compound to form a binding complex. After a predetermined period, the unbound VDR is removed. The VDR bound to the array can be quantified by adding an antibody to the VDR followed by the addition of a secondary antibody that is labeled. Test compound binding can be compared to a control binding partner, for example, 1,25(OH)₂D₃. If the amount of VDR binding on reaction sites containing a test compound is greater than the amount of VDR bound on a binding partner reaction site, then the test compound is a candidate compound for heart failure treatment. Since the location and identity of the test compounds are known, rapid identification of test compounds capable of specifically binding to VDR, for example, a nVDR or a VDRₜₜ can be achieved using this screening method.

Embodiments of the present technology are further illustrated through the following non-limiting examples.

### EXAMPLES

### Example 1

### Preparation of Rat Cardiomyocytes

Ventricular myocytes can be isolated from rat hearts as described previously [Westfall, et al, Methods Cell Biol. 52 (1997), pp. 307-322]. Female Sprage-Dawley rats (250-300 g) can be pre-treated with 0.01 U/kg heparin IP followed 10 minutes later by a lethal dose of pentobarbital. The heart is quickly removed and mounted on a Langendorff apparatus, and retrogradely perfused with isolation solution containing (in mmol/l): 130 NaCl, 5.4 KCl, 0.4 NaH₂PO₄, 1.4 MgCl₂.6H₂O, 0.5 CaCl₂, 10 HEPES, 10 glucose, 20 taurine, and 10 creatine, pH set to 7.4 using NaOH. When the coronary circulation has cleared of blood, perfusion is continued with Ca-free isolation solution (CaCl₂ replaced with 0.1 mM EGTA) for 4 min, followed by perfusion for a further 10 min with Ca-free isolation solution containing 0.8 mg/ml collagenase (type I; Worthington Biochemical, Lakewood, N.J.) and 0.1 mg/ml protease (type XIV; Sigma Chemical, St. Louis, Mo.). The ventricles were then excised, minced, and gently shaken at 37° C. in the collagenase-containing solution supplemented with 1% bovine serum albumin. Ventricular cells are filtered from this solution at 5 min intervals and resuspended in isolation solution containing 0.5 mmol/L Ca. All experiments are to be performed at room temperature (22-25° C.).

After isolation, cells can be pelleted by centrifugation at 50×g for 40 s and maintained under sterile conditions. The isolation solution is aspirated and replaced with DMEM supplemented with 5% FBS and 100 U/ml penicillin/streptomycin. Cells can be washed a further three times in culture medium before being plated out at a density of 104 cells/cm² on laminin coated coverslips and incubated at 37° C. under 5% CO₂. After 3 h non-adhering cells can be removed by careful aspiration and fresh culture medium can be added.

### Preparation of HL-1 Cells

HL-1 cells are obtained from Dr. W. C. Claycomb, Louisiana State University Medical Center, New Orleans, La. HL-1 cells (passages 55-75) are maintained at 37° C. under a 5% C0₂/air atmosphere in Claycomb medium (JRL Bioscience) supplemented with 10% fetal bovine serum, 0.29 mg/mL L-glutamine, 100 units/mL penicillin, 100 μg/mL streptomycin (all Gibco), and 0.1 mM norepinephrine (Sigma). The medium can be replaced approximately every 24 hours. HL-1 cells were grown in T25 culture flasks which were precoated overnight with 2 μg/cm fibronectin/0.02% gelatin solution. HL-1 cells were maintained until they reached confluence at which time the cells were removed by trypsinization and passed into another T25 culture flask coated with gelatin/fibronectin. Cells can be ultimately passed into T75 flasks to increase the number of cells prior to experimentation. Vitamin D experiments are performed in either T75 flasks, or Lab-Tech 2-well slides (for immunohistochemistry), 24 hours after a 1:2 split of confluent cells. For the time-dependant experiments 100 nM 1,25(OH)₂D₃ in 95% EtOH can be added to the media with 95% EtOH alone for control. The dose-response experiment can be carried out with a range of concentrations from 0.01 to 100 nM.

### Example 2

### Immunofluoresence of VDRₜₜ Containing Cells

Immunofluorescent staining of cells can be carried out in a method similar to that previously described (J. Huhtakangas, et al, Mol. Endocrinol. 18 (11) (2004) 2660-2671). Briefly, cells grown on coverslips were washed with PBS and fixed with 3.7% formaldehyde, 0.05% gluteraldehyde, and 0.5% Triton X100. Following blocking for 10 minutes in 10% goat serum/PBS the primary antibody was applied in 5% goat serum/PBS. After a one-hour incubation the slides are washed with PBS, incubated for 30 minutes with the FITC conjugated secondary antibody, and washed again. The cells are then stained for five minutes with DAPI (Invitrogen) and cover slips are mounted with Prolong Gold anti-fade reagent (Invitrogen). Slides can be analyzed with Olympus FV-500 on a Olympus iX 81 confocal microscope. Primary Ab's used can include sc1008 (anti-VDR C₂0), sc13133 (monoclonal anti-VDR D₆), and sc8094 (anti-SERCA2 C₆) all from Santa Cruz Biotechnology Co. (CA). Secondary Ab's include sc2024 and sc2783 (both Santa Cruz) and F8771 (Sigma).

Immunofluorescent staining of HL-1 cells can be carried out in a method similar to that previously described (Tevosian et al., 1999), except that 0.5% CHAPS (Fisher) can be used instead of Triton X-100. Briefly, HL-1 cells grown on 2-well slides (Lab-Tek) are initially preserved with Sprayfix cytology fixative. To prepare for staining, the slides are washed in EtOH and water, and then briefly washed with PBS. Cells were fixed with cold acetone, rinsed with PBS, and permeabilized by three ten minute washes with 0.5% CHAPS/PBS. Next, the cells are blocked overnight with 5% milk/10% goat serum/PBS. The permeabilization step is then repeated prior to incubation with the primary antibody in 10% goat serum/0.5% CHAPS/PBS with the antibodies and dilutions listed below. After a one-hour incubation with the primary antibodies, the slides are washed three times in the 10% goat serum/0.5% CHAPS/PBS, then three times in 0.5% CHAPS/PBS. Secondary antibodies, as described below, can be incubated for one-hour with 1% BSA/0.5% CHAPS/PBS. After washing with 0.5% CHAPS/PBS the cells are stained for five minutes with 4,6-diamidine-2-phenylidole-dihydrochloride (DAPI Nucleic Acid Stain; Invitrogen) and cover slips are mounted with Prolong Gold anti-fade reagent (Invitrogen). The following antibody combinations can be used: rabbit anti-mouse VDR (sc-1008; Santa Cruz Biotech; 1:100 dilution), rabbit anti-mouse ANP (AB 5490; Chemicon; 1:50 dilution), and rabbit anti-mouse c-myc (sc-764; Santa Cruz Biotech; 1:100 dilution), all followed by FITC conjugated goat anti-rabbit (AP 307F; Chemicon; 1:400). Mouse monoclonal anti-myotrophin (V 11220, BD Transduction Laboratory; 1:50) followed by FITC conjugated goat anti-mouse (F-0257; Sigma; 1:128), and goat anti-mouse calbindin-9 (sc-18038. Santa Cruz Bio; 1:100) followed by FITC conjugated rabbit anti-goat (AP 106F; Chemicon; 1:100). Slides can be analyzed on an Olympus BX 51 immunofluorescence microscope and photomicrographs can be recorded as digital images.

### Example 3

### Preparation and Isolation of Cell Membranes

Crude myocardial membranes were prepared as follows: whole hearts were removed from three month old female Sprague-Dawley rats and were cannulated and perfused two times with 10 mls ice-cold DPBS then once with 3 mls of ice-cold TKED lysis buffer (50 mM Tris-HCl, 150 mM KCl, 1.5 mM EDTA, 10 mM dithiothreitol, pH7.4 and a 1/100 dilution of Sigma Protease Inhibitor Cocktail [P8340]). Ventricles were cut away from atria and connective tissue and then minced, transferred to a 13 ml round-bottom Sarstedt tube and washed two times in 10 mls TKED lysis buffer by spinning briefly at full speed in a clinical centrifuge then aspirating. Tissue was kept ice-cold as much as possible. Washed minced ventricle tissue was then homogenized on ice with a Tekmar Tissuemizer in 5 volumes (˜5 mls) of TKED lysis buffer using 10 strokes at setting 30 followed by 5 strokes at setting 50. The resulting homogenate was transferred to a 15 ml Corex tube and centrifuged in a Beckman JA-20 rotor at 8,000 rpm (7,700 g) for 15 minutes at 4° C. The supernatant was transferred to a new 15 ml Corex tube and centrifuged in a Beckman JA-20 rotor at 18,200 rpm (40,000 g) for 25 minutes at 4° C. The resulting membrane pellet was washed once in a few mls TKED lysis buffer, resuspended in 0.5 mls TKED lysis buffer then homogenized with 3 strokes of a 2 ml Wheaton Potter-Elvejhem tissue grinder.

### Example 4

### Western Blot Analysis of Cardiomyocyte Membrane Preparations

Whole hearts are removed from 3 month old Sprague-Dawley female rats and are cannulated and perfused two times with 10 mls ice-cold DPBS then with 23 mls of ice-cold TKED lysis buffer (50 mM Tris-HCl, 150 mM KCl, 1.5 mM EDTA, 10 mM dithiothreitol, pH7.4 and a 1/100 dilution of Sigma Protease Inhibitor Cocktail [P8340]). Ventricles are cut away from atria and connective tissue and then minced and transferred to a 13 ml round-bottom Sarstedt tube. 18 inches of intestine from a rat can be removed, rinsed in cold water, then rinsed internally by injecting with DPBS. Mucousa was scraped off with a glass slide and transferred to a 13 ml round bottom Sarstedt tube. Both tissues can be washed two times in 10 mls TKED lysis buffer by spinning briefly at full speed in a clinical centrifuge then aspirating. Tissue is kept ice-cold as much as possible. Washed tissue is then homogenized on ice with a Tekmar Tissuemizer in 5 volumes (˜5 mls) of TKED lysis buffer using 10 strokes at setting 30 followed by 5 strokes at setting 50. The resulting homogenate (whole homogenate) is transferred to a 15 ml Corex tube and centrifuged in a Beckman JA-20 rotor at 8,000 rpm (7,700 g) for 15 minutes at 4° C. The pellet can be washed with a few mLs of TKED lysis buffer and then be resuspended in 2.5 mLs TKED lysis buffer (0-7,700 g pellet). The supernatant is transferred to a new 15 ml Corex tube and centrifuged in a Beckman JA-20 rotor at 18,200 rpm (40,000 g) for 25 minutes at 4° C. The resulting membrane pellet is washed once in a few mLs TKED lysis buffer then resuspended in 0.1 mL TKED lysis buffer to obtain a 7.7-40 kg pellet fraction. The supernatant can be transferred to a Beckman Ti 70.1 ultracentrifuge tube and centrifuged in a Beckman Ti 70.1 rotor at 35 k rpm for 1 hour at 4° C.

The resulting pellet is washed once in a few mLs TKED lysis buffer then resuspended in 0.1 mls TKED lysis buffer to obtain a 40-110 k g pellet fraction. The supernatant can be transferred to a 15 mL centrifuge tube and flash frozen (cytosol). Samples (100 μg) can be electrophoresed on a 10% Criterion SDS/PAGE Tris HCl gel (BioRad) and transferred to a PVDF membrane (Millipore, Bedford, Mass.). PVDF membranes can be incubated with a 1:100 fold dilution of primary antibody against VDR (C-20, Santa Cruz Biotechnology, Santa Cruz, Calif.) with 5% milk containing 0.1% Tween 20 for 1 hour at room temperature. After four 5 min rinses with TBST, membranes are incubated with a 1:1000 dilution of secondary antibody conjugated with horseradish peroxidase (G.E. Healthcare NA934V) for 2 hours at room temp. After four 5 min rinses, the membrane blots are incubated with Amersham ECL substrate and exposed to X-ray film.

### Example 5

### Vitamin D Binding Assays

Saturation binding assays with [³H] 1α,25(OH)₂ vitamin D₃ are done in quadruplicate in 250 μl of TKEDN binding buffer (50 mM Tris-HCl, 150 mM KCl, 1.5 mM EDTA, 10 mM dithiothreitol, 100 mM NaCl, pH7.4 and a 1/100 dilution of Sigma Protease Inhibitor Cocktail [P8340]) in 12×75 mm borosilicate glass tubes containing 20 μg membranes and concentrations of [³H] 1α,25(OH)₂ vitamin D₃ (specific activity 176 Ci/mmol) ranging from 0.0625 nM to 1 nM. Nonspecific binding can be determined in the presence of non-radiolabeled 1α,25(OH)₂ vitamin D₃ (2 μm). Assays are performed at room temperature for 60 minutes and can be filtered over glass fiber filters (Whatman GF-C) and washed three times with 5 mLs ice-cold TKEDN binding buffer. Filters are placed in 10 mLs UniverSol ES (MP Biochemicals) overnight then counted in a Beckman LS 5801 liquid scintillation counter. [³H] 1α,25(OH)₂ Vitamin D₃ can be obtained from G.E. Healthcare and is dried using a nitrogen evaporator and subsequently resuspended in ethanol. Non-radiolabeled 1α,25(OH)₂ vitamin D₃ can be obtained from Sigma-Aldrich, St. Louis, Mo., USA and is similarly diluted in ethanol.

Ligand binding assays to determine the ability of competitors or test compounds to compete with [³H] 1α,25(OH)₂ vitamin D₃ can be done in quadruplicate (for vehicle) or duplicate (for test compounds or competitors) in 250 μl of TKEDN binding buffer (50 mM Tris-HCl, 150 mM KCl, 1.5 mM EDTA, 10 mM dithiothreitol, 100 mM NaCl, pH7.4 and a 1/100 dilution of Sigma Protease Inhibitor Cocktail [P8340]) in 12×75 mm borosilicate glass tubes containing 20 μg membranes, 0.5 nM [³H] 1α,25(OH)₂ vitamin D₃ (specific activity 176 Ci/mmol) and various concentrations of competitor or test compound. Nonspecific binding is determined in the presence of cold 1α,25(OH)₂ Vitamin D₃ (2 μM) for vehicle and each competitor or test compound. Assays are performed at room temperature for 60 minutes. Reactants are then filtered over glass fiber filters (Whatman GF-C) and washed three times with 5 mls ice-cold TKEDN binding buffer. Filters are placed in 10 mLs UniverSol ES (MP Biochemicals) overnight then counted in a Beckman LS 5801 liquid scintillation counter. Tritiated [³H] 1α,25(OH)₂ vitamin D₃ can be obtained from G.E. Healthcare and dried using a nitrogen evaporator and resuspended in ethanol. 1α,25(OH)₂ Vitamin D₃ and competitors or test compounds can be obtained from Sigma-Aldrich and diluted in ethanol. Vitamin D₅ was obtained from the University of Illinois and was diluted in ethanol.

### Example 6

### Measurement of Cardiomyocyte Cell Number, Size and Morphology

HL-1 cells are grown in 10% FCS-supplemented medium or medium supplemented with 1% ITS and treated with either 0.1% EtOH (control vehicle), 1, 10 or 100 nM 1,25(OH) 2D3, 100 nM PMA or 1 mM PMA or 1 mM 24,25(OH) 2D3 or 25(OH)D3 for 2 or 4 days. At each time point the myocytes are removed from the dish using a Trypsin-EDTA solution (Sigma Chemical, St. Louis, Mo.) and counted using a Coulter Counter (Model ZF, Coulter Electronics, Hialeah, Fla.). Protein levels can be determined after 2 days using the Bradford protein assay (14). HL-1 cells can then be prepared for flow cytometry by washing the cells 2× with phosphate buffered saline (PBS, Sigma Chemical, St. Louis, Mo.). The myocytes are then suspended in standard azide buffer (PBS with 1% FCS and 0.1% NaN3) and fixed with EtOH. The myocytes are then washed 2× with standard azide buffer and resuspended in 200 ul citrate buffer containing RNase A (38 mM trisodium citrate, 7.14 g/L RNase A) and incubated at 37° C. for 30 min. An equal volume propridium iodide solution (38 mM trisodium citrate, 0.05 g/L propridium iodide, pH 8.4) can be added and the myocytes stained for 30 min at 37° C., and stored at 4° C. in the dark until analysis. The HL-1 cells can then be examined on a flow cytometer (Coulter Elite, Coulter Electronics, Hialeah, Fla.) and cell cycle distribution can be determined by using MODFIT (v. 5.2).

### Western Blot Analysis

Treated and control samples of HL-1 cells are trypsinized and removed from flasks. Cell counts are performed under microscope with a Fisher hemacytometer. Samples are centrifuged at 3000 RPM for 2 minutes to obtain a pellet, which is then re-suspended in an SDS-leupeptin sample buffer (SB). This whole cell lysate can then be boiled for 15 minutes, separated into working aliquots, and stored at −20° C. for analysis. Standard Bradford Protein Determination is then performed on all samples. Samples are then electrophoresed as described in Example 4. Following electrophoresis, protein is transferred onto a PVDF (Millipore) membrane. After blocking the membrane for one hour with 5% milk in TBS containing 0.05% Tween 20 (Sigma), the blots are incubated with primary and secondary antibodies according to the dilutions listed below. The blot is washed three times in TBS/0.5% Tween 20 after each incubation, followed by one final wash in TBS. The membrane can then be treated with ECL developer (Amersham Biosciences) and exposed to Kodak Biomax XAR film. To confirm equal loading of protein into the wells each blot can be stripped and re-probed with HRP conjugated goat anti-mouse actin antibody (sc-1616HRP; Santa Cruz Biotechnology Co; 1:1000). The primary antibodies that can be used include rabbit anti-mouse VDR (sc-1008; Santa Cruz Bio; 1:200 dilution); rabbit anti-mouse ANP (AB 5490; Chemicon; 1:500 dilution), mouse monoclonal anti-myotrophin (V11220; BD Transduction Laboratory; 1:1000), rabbit anti-mouse c-myc (sc-764, Santa Cruz Bio; 1:100), and goat anti-mouse calbindin-9 (sc-18038, Santa Cruz Bio; 1:100). The secondary antibodies which can be used include HRP conjugated goat anti-rabbit (AP 132P, Chemicon; 1:1000) for VDR, ANP, and c-myc. HRP conjugated goat anti-mouse (A 2304, Sigma; 1:1000), and bovine anti-goat Ab (sc2350, Santa Cruz Bio; 1:250), for myotrophin and calbindin-9, respectively.

### Immunohistochemistry

Immunofluorescent staining of HL-1 cells can be carried out in a method similar to that previously described (Tevosian et al., 1999), except that 0.5% CHAPS (Fisher) can be used instead of Triton X-100. Briefly, HL-1 cells grown on 2-well slides (Lab-Tek) are initially preserved with Sprayfix cytology fixative. To prepare for staining, the slides are washed in EtOH and water, and then briefly washed with PBS. Cells were fixed with cold acetone, rinsed with PBS, and permeabilized by three ten minute washes with 0.5% CHAPS/PBS. Next, the cells are blocked overnight with 5% milk/10% goat serum/PBS. The permeabilization step is then repeated prior to incubation with the primary antibody in 10% goat serum/0.5% CHAPS/PBS with the antibodies and dilutions listed below. After a one-hour incubation the slides are washed three times in the 10% goat serum/0.5% CHAPS/PBS, then three times in 0.5% CHAPS/PBS. Secondary antibodies, as described below, can be incubated for one-hour with 1% BSA/0.5% CHAPS/PBS. After washing with 0.5% CHAPS/PBS the cells are stained for five minutes with 4,6-diamidine-2-phenylidole-dihydrochloride (DAPI Nucleic Acid Stain; Invitrogen) and cover slips are mounted with Prolong Gold anti-fade reagent (Invitrogen). The following antibody combinations can be used: rabbit anti-mouse VDR (sc-1008; Santa Cruz Biotech; 1:100 dilution), rabbit anti-mouse ANP (AB 5490; Chemicon; 1:50 dilution), and rabbit anti-mouse c-myc (sc-764; Santa Cruz Biotech; 1:100 dilution), all followed by FITC conjugated goat anti-rabbit (AP 307F; Chemicon; 1:400). Mouse monoclonal anti-myotrophin (V 11220, BD Transduction Laboratory; 1:50) followed by FITC conjugated goat anti-mouse (F-0257; Sigma; 1:128), and goat anti-mouse calbindin-9 (sc-18038. Santa Cruz Bio; 1:100) followed by FITC conjugated rabbit antigoat (AP 106F; Chemicon; 1:100). Slides can be analyzed on an Olympus BX 51 immunofluorescence microscope and photomicrographs can be recorded as digital images.

The description of the invention is merely exemplary in nature and, thus, variations that do not depart from the gist of the invention are intended to be within the scope of the invention. Such variations are not to be regarded as a departure from the spirit and scope of the invention.

1. A method for screening for a test compound which modulates the activity of a VDR for the treatment of heart failure, wherein the method comprises:
(a) contacting a test compound with VDR in a reaction mixture, wherein the reaction mixture conditions permits the test compound and a labeled binding partner to bind to the VDRₜₜ binding site and form a labeled binding complex;
(b) determining the level of labeled binding complex in the presence of the test compound; and
(c) comparing the level of the labeled binding complex in the presence of the test compound and labeled binding partner to the level of labeled binding complex in the presence of the labeled binding partner and in the absence of the test compound, wherein a decreased formation of labeled binding complex in the presence of the test compound indicates that the test compound is a heart failure candidate compound.

2. The method of claim 1, wherein the VDR is nuclear VDR, t-tubule VDR (VDRₜₜ) or both.

3. The method of claim 1, wherein the labeled binding partner is 1α,25(OH)₂ Vitamin D₃ labeled with a radioisotope, a fluorescence label, a chemiluminescent label, a conjugated enzyme, biotin, histidine peptide, avidin, and combinations thereof.

4. The method of claim 1, wherein the labeled binding complex is separated from unbound test compound and labeled binding partner prior determining the level of labeled binding complex, whereby the level of labeled binding complex is determined by measuring the bound labeled binding partner in the labeled binding complex.

5. A method for screening candidate compounds for the treatment of heart failure comprising:
(a) contacting a test compound to a reaction mixture, the reaction mixture comprising:
(i) a VDR polypeptide,
(ii) a Protein Kinase C polypeptide
(iii) a cell extract comprising radiolabeled ATP and a PKC specific substrate, wherein the reaction mixture conditions permit binding of the VDR to the cell extract containing Protein Kinase C to phosphorylate the PKC specific substrate;
(b) detecting levels of formation of the phosphorylated PKC specific substrate in the reaction mixture in the presence of the test compound; and
(c) measuring the amount of Protein Kinase C activity in the presence of the test compound and of a control, wherein an increase in the amount of Protein Kinase C activity in the presence of the test compound as compared to the control indicates that the test compound is a heart failure candidate compound.

6. The method of claim 5, wherein the PKC specific substrate is phospholamban, cardiac troponin I, H-Arg-Arg-Gly-Arg-Thr-Gly-Arg-Gly-Arg-Arg-Gly-Ile-Phe-Arg-OH (SEQ ID NO. 1), any peptide having a serine or threonine residue or combinations thereof.

7. The method of claim 5, wherein the VDR polypeptide contacted with a test compound of step (a) is isolated from a cardiomyocyte, an epithelial cell, a myocyte, and combinations thereof by isolating the VDR polypeptide from the cytosol or the plasma membrane of the cardiomyocyte, an epithelial cell, a myocyte.

8. The method of claim 5, wherein the Protein Kinase C polypeptide is incubated in step (a) in the form of PKC α, PKC δ, PKC ε and PKC γ.

9. The method of claim 5, wherein the test compound, VDR polypeptide and cell extract are incubated for at least about 3 minutes to about 60 minutes.

10. A method of screening for test compounds that are heart failure candidate compounds, the method comprising:
contacting cardiomyocyte cells having a mature cardiac myocyte phenotype with a test compound;
determining the level of cellular expression of Protein Kinase C or Protein Kinase A isoforms in the presence of the test compound; and
comparing the resulting determined level with a reference level determined, under the same conditions, for cellular expression of Protein Kinase C isoforms in the absence of the test compound,
wherein an equivalent or an increase in the cellular expression of Protein Kinase C isoforms or Protein Kinase A in the presence of the test compound as compared to the reference indicates that the test compound is a heart failure candidate compound.

11. The method of claim 10, wherein the cardiomyocyte cells are prepared from mammalian adult hearts, mammalian neonatal hearts or cultured immortalized cardiac myocyte tumor cells.

12. The method of claim 11, wherein the cardiomyocyte are prepared from perfused ventricular tissue.

13. The method of claim 11, wherein the immortalized cardiac myocyte tumor cells are HL-1 cells.

14. The method of claim 10, wherein the cellular expression of Protein Kinase C isoform is determined by radioimmunoassay, immunofluorescence, western blotting, quanitative RT-PCR, Northern blotting and immunoprecipitation.

15. The method of claim 10, wherein the PKC isoforms include one or more of PKC α, PKC δ, PKC ε and PKC γ.

16. The method of claim 10, further comprising detecting the level of expression of PKC isofoms by measuring the level of phosphorylation of phospholamban or cardiac troponin I present intracellularly after contacting a cardiomyocyte with a test compound and reference.

17. The method of claim 10, wherein the reference is 1α,25(OH)₂ Vitamin D₃.

18. A method for screening for a candidate compound that modulates the activity of a Vitamin D receptor for the treatment of heart failure, the method comprising:
(a) contacting a cell having a Vitamin D receptor with a first sample comprising a test compound, thereby forming a treated cell, and thereafter observing one or more phenotypic parameters thereof selected from the group consisting of cell size, cell proliferation, and cell morphology and combinations thereof;
(b) contacting under the same conditions as used in (a), a cell having a Vitamin D receptor with a second sample identical in composition to the first sample minus the test compound, thereby forming a control cell, and thereafter observing one or more phenotypic parameters thereof selected from the group consisting of cell size, cell proliferation, and cell morphology and combinations thereof; and
(c) comparing the observed phenotypic parameters to find a significant difference between said treated and control cells; and
(d) verifying that the test compound is a ligand for the Vitamin D receptor; and
(e) identifying, based on a significant difference found in (c), the test compound as a candidate compound that modulates the activity of the Vitamin D receptor for the treatment of heart failure.

19. The method of claim 18, wherein the cell is a cell having a functional VDR in the plasma membrane or in the cytosol.

20. The method of claim 19, wherein the cell is a mammalian cardiomyocyte, HL-1, a eukaryotic cell transfected with a nuclear or membrane VDR encoding nucleic sequence and combinations thereof.

21. The method of claim 19, wherein the cell is HL-1.

22. The method of claim 18, wherein cell size is observed by comparing the size of the cells microscopically.

23. The method of claim 18, wherein cell proliferation is observed by counting an average number of cells present in a microscopic field multiplied by the volume of cells contained within one microscopic field to yield a number of cells per milliliter.

24. The method of claim 18, wherein the cell morphology is observed by placing one or more cells after contact with the first or second samples in a microscope slide under magnification sufficient to visually record the differences in the morphology of the cell as compared to cells the cells prior to contact with sample 1 or 2.

25. The method of claim 18, wherein the test compound is verified as a VDR binding compound by binding the test compound to cell extracts containing functional VDR and competing with radiolabelled 1α,25(OH)₂ Vitamin D₃, wherein displacement of radiolabelled 1α,25(OH)₂ Vitamin D₃ by the test compound verifies the test compound can bind to VDR.

26. A method for screening for a candidate compound that modulates the activity of a Vitamin D receptor for the treatment of heart failure, the method comprising:
(a) contacting a cell having a vitamin D receptor and expressing at least one biomarker for cardiac hypertrophy with a test compound in a diluent, to form a test cell;
(b) contacting the same cell type as in step (a) with the diluent in the absence of the test compound, to form a control cell; and
(c) verifying that the test compound is a ligand for the Vitamin D receptor; and
(d) comparing the level of expression of the biomarker in the test and control cells to identify a significant difference therein;
(e) identifying based on a significant difference found in (d), the test compound as a candidate heart failure compound.

27. The method of claim 26, wherein the cell is a mammalian cardiomyocyte, HL-1, myocyte, and combinations thereof.

28. The method of claim 26, wherein the expression of the cellular biomarker comprises measuring the cellular level of any one or more of c-myc, myotrophin, phospholamban, PKC, PKA, ANP, PCNA, calbindin 9 in the test cell and control cell.
